(19) United States
(12) Patent Application Publication
(10) Pub. No.: US 2007/0270484 A1
(43) Pub. Date: Nov. 22, 2007

(54) 3'-N-Substituted-3-O-Substituted Erythronolide a Derivatives

(76) Inventors: Biswajit Das, Haryana (IN); Mohammad Salman, Princeton, NJ (US); Sachin Vilasrao Shelke, Maharastra (IN); Atul Kashinath Hajare, Haryana (IN); Sanjay Talukdar, West Bengal (IN); Sujata Rathy, Delhi (IN); Arani Pal, West Bengal (IN); Ashok Rattan, Delhi (IN)

Correspondence Address: RANBAXY INC., 600 COLLEGE ROAD EAST, SUITE 2100, PRINCETON, NJ, 08540, US

(21) Appl. No.: 10/573,275

(22) PCT Filed: Sep. 25, 2003
(86) PCT No.: PCT/IB03/04166

§ 371 Date: Apr. 11, 2007

(51) Int. Cl. A61K 31/7048; A61P 31/04; C07H 17/08

(52) U.S. Cl. 514/450; 549/271

(57) ABSTRACT

This invention generally relates to macrolides, more particularly, the invention relates to 3-N-substituted-3-O-substituted erythronolide A derivatives, which are antibacterial agents effective against gram positive or gram negative bacteria and atypical pathogens. The compounds of this invention are more particularly effective against Staphylococcus aureus, Streptococcus pneumoniae, Enterococcus faecalis, Escherichia coli, Pseudomonas aeruginosa, Haemophilus influenzae. The invention also relates to a process for the preparation of the compounds of the present invention, pharmaceutical compositions containing the compounds of the present invention and the methods for treating bacterial infection.

### FIELD OF INVENTION

This invention generally relates to macrolides, more particularly, the invention relates to 3′-N-substituted-3-O-substituted erythronolide A derivatives, which are antibacterial agents effective against gram positive or gram negative bacteria and atypical pathogens. The compounds of this invention are more particularly effective against Staphylococcus aureus, Streptococcus pneumoniae, Enterococcus faecalis, Escherichia coli, Pseudomonas aeruginosa, Haemophilus influenzae. The invention also relates to a process for the preparation of the compounds of the present invention, pharmaceutical compositions containing the compounds of the present invention and the methods for treating bacterial infection.

### BACKGROUND OF THE INVENTION

The first generation macrolides erythromycin A and the early derivatives are characterized by bacteriostatic or bacteriocidal activity for most gram-positive bacteria, atypical pathogens, many communities acquired respiratory infections, in-patients with penicillin allergy. However, erythromycin A causes numerous drug-drug interactions, has relatively poor absorption, poor local tolerance, loses its antibacterial activity under acidic conditions by degradation and the degraded products are known to be responsible for undesired side effects. (Itoh, Z., et al., Am. J. Physiol., (1984), 247: 688; and Omura, S., et al., J. Med. Chem., (1987), 30, 1943). Various erythromycin A derivatives have been prepared to overcome the acid instability and other problems associated with it.

Roxithromycin, clarithromycin (6-O-methylerythromycin A) and azithromycin (azalides) have been developed to address the limitation of erythromycin A. Both clarithromycin and azithromycin have proved to be important drugs in the treatment and prophylaxis of atypical mycobacterial infectious in-patient with HIV.

Macrolides have proved to be effective drugs in the treatment of many respiratory tract infections. However, increasing resistance among S. pneumoniae has prompted the search for new compounds that retain the favourable safety profile, and a spectrum of activity confined to respiratory pathogens (Expert Opinion Investigations Drugs, (2001), 10, 353-367). Consequently, numerous investigators have prepared chemical derivatives of erythromycin A in an attempt to obtain analogs having modified or improved profiles of antibiotic activity. It is reported that 6-O-methyl erythromycin A derivatives have improved acid stability and have superior in vivo antibacterial activity in comparison with erythromycin A when administered orally (Morimeto, et al., Antibiotic, (1984), 37, 187; J. Antibiotic, (1990), 43, 286; EP Pat. Nos. 272,110, and 215,355; and U.S. Pat. No. 4,331,803). It is reported that 6-O-methyl erythromycin derivatives and their 11,12-cyclic carbamate derivatives have a superior in vivo antibacterial activity as well as stability to acids (U.S. Pat. No. 4,742,049; and EP Pat. No. 487,411). 6-O-methyl-3-descladinose erythromycin A (as in WO 97/10251), 6-O-substituted-3-oxoerythromycin A (as in U.S. Pat. No. 5,444,051), 3-deoxy-3-descladinose erythromycin A (as in WO 97/17356), tricyclic erythromycin A (as in WO 92/09614) and bicyclic 6-O-methyl 3-oxoethythromycin A (as in EP. Pat. No. 596,802) derivatives have been reported. There are also reports relating to ester derivatives at the C-3 position (EP. Pat. No. 619,320) with an enhanced spectrum of antibacterial activity. Ketolides exhibits greater efficacy and safety, has broader spectrum of activities, and is particularly effective against resistant pathogens, have been developed as next generation macrolides. 3′-N-modified-6-O-substituted erythromycin ketolides have been described (U.S. Pat. No. 6,034,69). Telithromycin (Aventis) and ABT-773 (Abbott) are expected to receive the FDA approval. They appeared to represent a useful alternative to macrolides and quinolones for treatment of community acquired respiratory infections.

Despite these advances, a need for better profile of antibiotic activity remains. Ideally, such derivatives would be water soluble with oral efficacy. Such compounds would provide useful agents for bacterially infectious diseases. There is no example available in the prior art wherein the compounds of the present invention, containing 3′-N-substituted-3-O-substituted erythronolide A derivatives are useful as therapy for curing bacterial infections.

### SUMMARY OF THE INVENTION

Novel 3′-N-substituted-3-O-substituted erythronolide A derivatives are provided, which are useful for the safe treatment of bacterially infectious disease, and methods for the syntheses of these compounds are also provided. The pharmaceutically acceptable acid addition salts, pharmaceutically acceptable solvates, enantiomers, diastereomers, polymorphs of these compounds, as well as metabolites having same type of activity are also provided. Pharmaceutical compositions containing such compounds their pharmaceutically acceptable acid addition salts, pharmaceutically acceptable solvates, enantiomers, diastereomers, polymorphs and metabolites, together with acceptable carriers, excipients or diluents, which are useful for the treatment of bacterially infectious disease are also provided.

The compounds disclosed herein were screened for antibacterial activity in vitro using agar incorporation method. Several compounds exhibited significant antibacterial activity. The 3′-N-substituted-3-O-substituted erythronolide A derivatives are useful as therapy for curing bacterial infections.

Other aspects will be set forth in the description, which follows and in part will be apparent from the description or may be learnt by the practice of the invention. Advantages which derive from the compounds and methods disclosed herein may be realized and obtained by means of the mechanism and combination pointed out in the appended claims.

In one aspect, there are provided compounds having structures of Formula I: 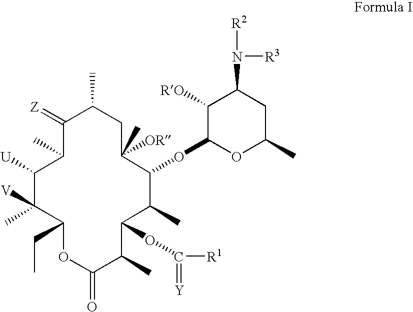
and their pharmaceutically acceptable acid addition salts, pharmaceutically acceptable solvates, enantiomers, diastereomers, polymorphs and metabolites, wherein:

R¹ represents: lower alkyl (C₁-C₅) group, lower alkyl (C₁-C₅) having one or more halogen (F, Cl, Br, I) atoms as substituent (s), lower alkyl (C₁-C₅) amino group, lower alkyl amino (C₁-C₅) carbonyl group; lower alkoxy group (C₁-C₅); or five or six membered aryl or heteroaryl ring having 1 to 3 hetero atoms such as oxygen, nitrogen, and sulphur, wherein the aryl or heteroaryl ring may be unsubstituted or substituted by 1 to 3 substituents such as lower alkyl (C₁-C₅) group, lower alkyl (C₁-C₅) group having one or more halogen atoms, lower alkoxy (C₁-C₅) groups, lower alkyl (C₁-C₅) amino group, halogen atoms, amino group, nitro group, hydroxy group, and cyano group;

R² and R³ are independently selected from: C₁-C₆ alkyl group optionally substituted with halogen atoms; cycloalkyl (C₃-C₇) group; or five to six membered aryl or heteroaryl ring having 1 to 3 hetero atom such as nitrogen, oxygen, and sulphur, wherein the aryl or heteroaryl ring may be unsubstituted or substituted by 1 to 3 substituents such as lower alkyl (C₁-C₃), lower alkyl (C₁-C₃) group having one or more halogen atom as substituent(s), lower alkoxy (C₁-C₃) group, lower alkyl (C₁-C₃) amino group, halogen (F, Cl, Br, I) atoms, nitro group, hydroxy group, and cyano group; the above-mentioned C₁-C₆ alkyl group may be substituted by: NHCOR⁵, NHCOOR⁵, OCOR⁵, or COR⁵ wherein R⁵ represents lower alkyl (C₁-C₅); five to six membered aryl or heteroaryl ring having 1 to 3 hetero atom such as nitrogen, oxygen, and sulphur, wherein the aryl or heteroaryl ring may be unsubstituted or substituted by 1 to 3 substituents such as lower alkyl (C₁-C₃), lower alkyl (C₁-C₃) group having one or more halogen atoms as substituent(s), lower alkoxy (C₁-C₃) group, lower alkyl (C₁-C₃) amino group, halogen atoms, nitro group, hydroxy group, and cyano group; C₂-C₆ alkenyl or alkyne group optionally substituted with halogen atoms or a group such as NHCOR⁵, NHCOOR⁵, COR⁵, or OCOR⁵ (wherein R⁵ is as defined above); cycloalkyl (C₃-C₇) group; five or six membered aryl or heteroaryl ring having 1 to 3 hetero atom independently selected from the group consisting of nitrogen, oxygen, and sulphur, wherein the aryl or heteroaryl ring may be unsubstituted or substituted by 1 to 3 substituents independently selected from the group consisting of lower alkyl (C₁-C₃) group, lower alkyl (C₁-C₃) group having one or more halogen atoms as substituent(s), lower alkoxy (C₁-C₃) group, lower alkyl (C₁-C₃) amino, halogen (F, Cl, Br, I) atoms, nitro group, hydroxy group, amino group, and cyano group;

R′ represents hydrogen, or a hydroxy protecting group such as acetyl, benzoyl, butyldiphenylsilyl, methylthiomethyl, or methoxy methyl;

R″ represents hydrogen, or a lower alkyl (C₁-C₃) group;

Y represents oxygen or sulphur;

Z represents an oxygen atom or a group represented by NOR⁶, wherein R⁶ represents hydrogen atom, alkyl (C₁-C₆) group, alkyl (C₁-C₆) amino group, phenyl or benzyl group, or phenyl or benzyl group having 1 to 5 substituents such as halogen atoms, lower alkyl (C₁-C₃) group, hydroxy group, nitro group, cyano group, or amino group;

U represents a hydroxy group, OR⁷, wherein R⁷ represents hydroxy protecting group such as acetyl, benzoyl, butyldiphenylsilyl, methylthiomethyl, or methoxymethyl; or —NH(CH₂)ₙR⁸, wherein n represents 0 to 4 and R⁸ represents five or six membered aryl or heteroaryl ring having 1 to 4 hetero atom such as nitrogen, oxygen, and sulphur, wherein the aryl or heteroaryl ring may be unsubstituted or substituted by one to three substituents such as lower alkyl (C₁-C₃) group, lower alkyl (C₁-C₃) group having one or more halogen atoms as substituent(s), lower alkoxy (C₁-C₃) group, lower alkyl (C₁-C₃) amino, halogen atoms, nitro group, hydroxy group, amino group, and cyano group;

V represents: hydrogen atom; hydroxy group; or OR⁷, wherein R⁷ represents a hydroxy protecting group such as acetyl, benzoyl, butyldiphenylsilyl, methylthiomethylmethyl and methoxymethyl;

U and V may also together represent (with carbon atoms at the 11- and 12-positions on the erythronolide skeleton): a group represented by Formula 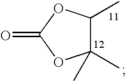
or a group represented by the Formula 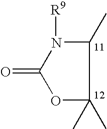
wherein R⁹ represents: hydrogen atom; alkyl (C₁-C₆) group, wherein the alkyl (C₁-C₆) may be unsubstituted or substituted by halogen atoms, five or six membered aryl or heteroaryl ring having 1 to 3 hetero atoms such as nitrogen, oxygen, and sulphur, wherein the aryl or heteroaryl ring may be unsubstituted or substituted by 1 to 3 substituents such as lower alkyl (C₁-C₃) group, lower alkyl (C₁-C₃) group having one or more halogen (F, Cl, Br, I) atoms as substituent(s), lower alkoxy (C₁-C₃) group, lower alkyl (C₁-C₃) amino, halogen (F, Cl, Br, I) atoms, nitro group, hydroxy group, amino group, and cyano group.

In a second aspect, there is provided a method of treating or preventing an animal or human suffering from bacterial infection comprising administering to a patient in need thereof, a therapeutically effective amount of a compound as described above.

In a third aspect, there is provided a method of treating or preventing an animal or human suffering from bacterial infection mediated through Staphylococcus aureus, Streptococcus pneumoniae, Enterococcus faecalis, Escherichia coli, Pseudomonas aeruginosa, or Haemophilus influenzae. with a compound as described above.

In a fourth aspect, there is provided a method of treating or preventing an animal or human suffering from bacterial infection, comprising administering to a patient in need thereof, a therapeutically effective amount of a pharmaceutical composition including a compound as described above.

In a fifth aspect, there is provided a method of treating or preventing an animal or human suffering from bacterial infection mediated through Staphylococcus aureus, Streptococcus pneumoniae, Enterococcus faecalis, Escherichia coli, Pseudomonas aeruginosa, or Haemophilus influenzae with a therapeutically effective amount of a pharmaceutical composition as described above.

In a sixth aspect, there are provided processes for preparing 3′-N-substituted-3-O-substituted erythronolide A derivatives.

The compounds described herein exhibit significant potency in terms of their antibacterial activity in vitro. Therefore, pharmaceutical compositions and methods for treatment of bacterially infectious diseases are provided herein. In addition, the compounds or pharmaceutical compositions disclosed herein can be administered orally or parentally.

### DETAILED DESCRIPTION OF THE INVENTION

The compounds described herein may be prepared by the following reaction sequence.

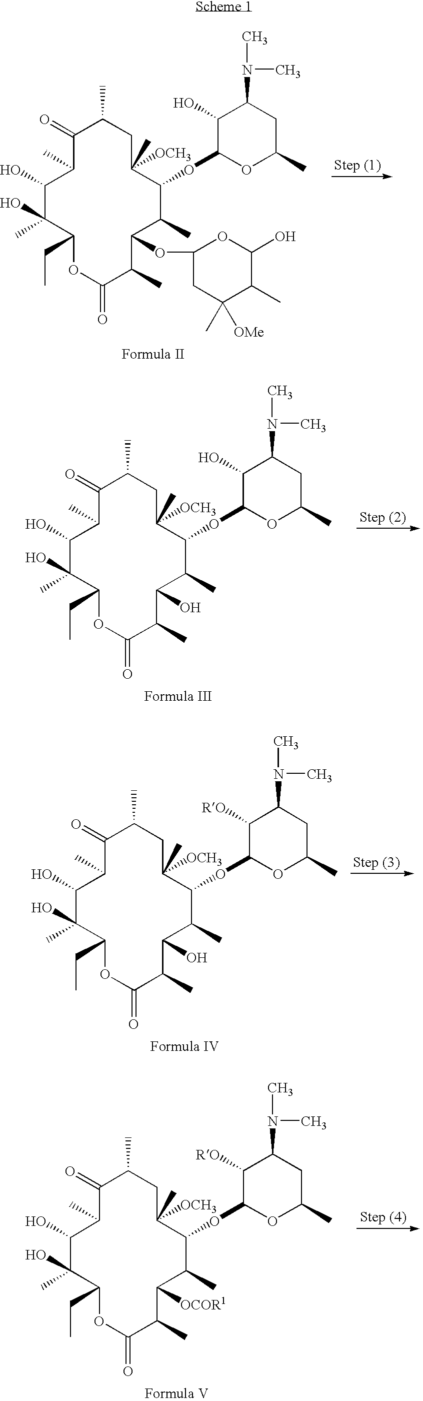

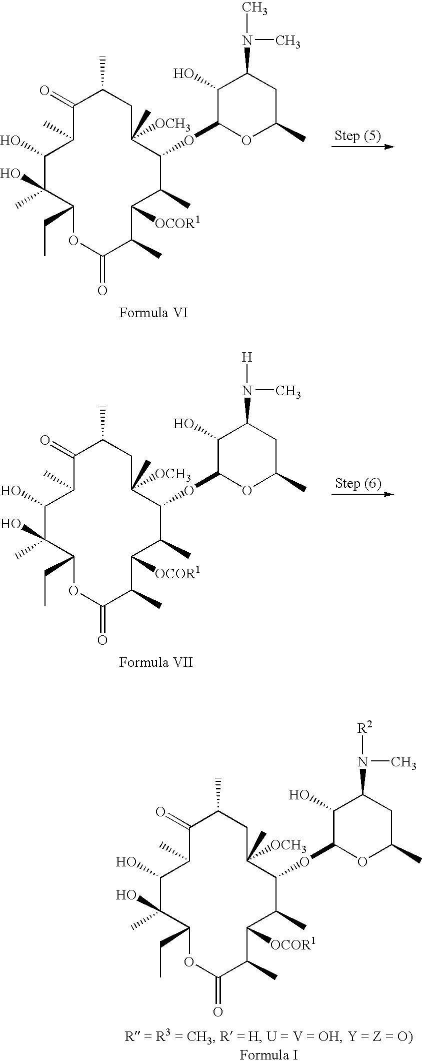

In accordance with Scheme 1, clarithromycin of Formula II is converted into particular desired compounds of Formula I by a series of steps as follows:

Step (1): Clarithromycin of Formula II is suspended in aqueous alcohol such as methanol, ethanol, propanol, or isopropanol, and is hydrolyzed with a mineral or organic acid such as hydrochloric acid or dichloroacetic acid at an ambient temperature for about 1 to 25 hours to give a compound of Formula III.

Step (2): The compound of Formula III is reacted with R′₂O or R′X (where R′ is a hydroxy protecting group such as acetyl, benzoyl, butyldiphenylsilyl, methylthiomethyl, or methoxymethyl, and X is a halogen atom) in the presence of an inorganic base such as sodium hydrogen carbonate, or potassium carbonate or an organic base such as triethylamine, pyridine, tributylamine, or 4-N-dimethylaminopyridine, in an inert solvent such as dichloromethane, dichloroethane, acetone, ethyl acetate, or tetrahydrofuran, at a temperature of from 0° C. to 30° C., to give a compound of Formula IV.

Step (3): The compound of Formula IV is reacted with R¹COOH, R¹COX, (R¹CO)₂O or R¹COOR⁴ (where R¹ is as defined for Formula I and R⁴ is a group such as pivaloyl group, p-toluenesulfonyl group, isobutoxycarbonyl group, ethoxycarbonyl group or isopropoxycarbonyl group) and activating reagent such as dicyclohexylcarbodiimide (DCC) or 1-ethyl-3(3-dimethylaminopropyl)carbodiimide hydrochloride (EDCI) in the presence of an inorganic base such as sodium hydrogen carbonate, or potassium carbonate, or an organic base such as triethylamine, pyridine, tributylamine, or 4-dimethylaminopyridine, in an inert solvent such as dichloromethane, dichloroethane, acetone, ethyl acetate, or tetrahydrofuran, at a temperature of from 0° C. to 30° C. to give a compound of Formula V.

Step (4): The compound of Formula V is heated with alcohol as used in step (1), at a temperature of from 30° C. to 100° C. for about 5 to 25 hours to remove the protecting group (R′) at the 2′-position of desosaminyl moeity, to obtain a compound of Formula VI.

Step (5): The compound of Formula VI is desmethylated at 3′-N-dimethyl group with N-iodosuccinamide and acetonitrile or iodine in the presence of a base such as sodium acetate followed by quench with sodium thiosulfonate to give a compound of Formula VII. Alternatively, the reaction is carried out using reagents such as benzylchloroformate, allylchloroformate, or vinylchloroformate.

Step (6): The compound of Formula VII is reacted with a reagent represented by the Formula R²CHO (or its precursor), R²₂CO or R²X (wherein R² is as defined for Formula I and X represents halogen atom) in the presence of a reducing agent such as sodium borohydride, sodium cyanoborohydride or sodium triacetoxyborohydride or in the presence of palladium and carbon catalyst in a protic or non-protic solvent such as hexane, toluene, methylene chloride, ethylene chloride, chloroform, tetrahydrofuran, N-methyl-pyrrolidinone, diethyl ether, bis-methoxymethyl ether, dimethylformamide, acetonitrile, acetone, or ethyl acetate, under hydrogen atmosphere to give a particular compound of Formula I (wherein R³═R″═CH₃, R′═H, U═V═OH, and Y=Z=O).

The order of steps presented in Scheme I is not critical, and reactions in other orders can also be carried out to produce particular compounds of Formula I. For example, step (5) of Scheme I, desmethylation at the 3′-N-dimethyl group, can be carried out as a first step on clarithromycin of Formula II, to produce an N-desmethylated compound of Formula VIII 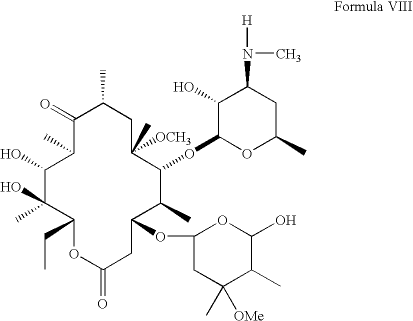
with reagents and conditions as described above for Scheme I, step (5). This can be followed by introduction of R² (step (6) in Scheme I), to produce a compound of Formula IX 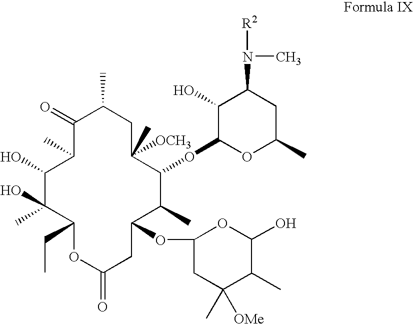
using reagents and conditions as described above for Scheme I, step (6). This can be then followed by hydrolysis of a compound of Formula IX to produce a compound of Formula X 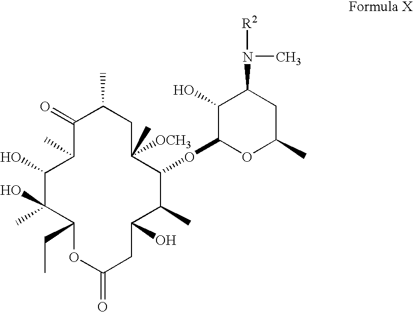
using reagents and conditions as described above for Scheme I, step (1). Reaction of a compound of Formula X with a reagent R′₂O or R′X (R′ and X as previously described) can then result in a compound of Formula XI using reagents and conditions as described above for Scheme I, step (2). 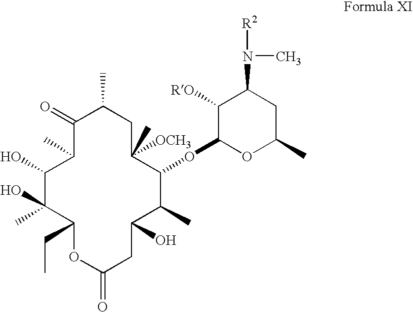
Reaction of a compound of Formula XI with reagent R¹COOH, R¹COX, (R¹CO)₂O or R¹COOR⁴ (R¹, X and R⁴ as described above) can produce a compound of Formula XII using reagents and conditions as described above for Scheme I, step (3). 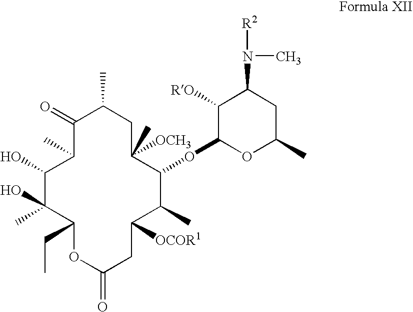
Deprotection of a compound of Formula XII can produce a compound of Formula I, using reagents and conditions as described for Scheme I, step (4).

In another example, steps (1) through (3) can be carried out on clarithromycin of Formula II to produce compound of Formula V. From this point, desmethylation of the compound of Formula V can produce a compound of Formula XIII 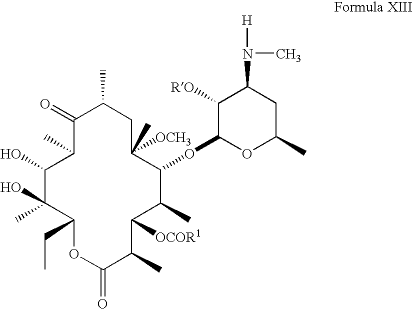
using reagents and conditions as described above for Scheme I, step (5). Deprotection of a compound of Formula XIII can produce a compound of Formula VII, using reagents and conditions as described for Scheme L step (4). Then, introduction of R², as in Scheme I, step (6), can produce a compound of Formula I, using the described reagents and conditions.

In yet another example, steps (1) and (2) can be carried out on clarithromycin of Formula II to produce a compound of Formula IV. From this point, desmethylation of the compound of Formula IV can be carried out to produce a compound of Formula XIV. 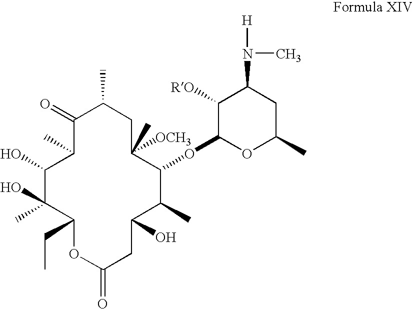
Introduction of R², as in Scheme I, step (6), can produce compound of Formula XI, using the described reagents and conditions. Reaction of a compound of Formula XI with reagent R¹COOH, R¹COX, (R¹CO)₂O or R¹COOR⁴ (R¹, X and R⁴ as described above) can produce a compound of Formula XII using reagents and conditions as described above for Scheme I, step (3). Deprotection of a compound of Formula XII can produce a compound of Formula I, using reagents and conditions as described for Scheme I, step (4).

Other feasible combinations of steps will be recognized by those of ordinary skill in the art, using reagents described herein, or appropriate alternative regants known in the art for these transformations.

Preferred compounds according to the invention and capable of being produced by Scheme 1 include:

| Compound | |
|---|---|
| No. | Chemical Name |
| 1. | 3-O-(3-Fluorophenyl)acetyl-5-O-(3′-N-desmethyl-3′-N- |
| | cyclopropylmethyl)desosaminyl erythronolide A |
| | (Compound No. 1) |
| 2. | 3-O-(2-Fluorophenyl)acetyl-5-O-(3′-N-desmethyl-3′-N- |
| | cyclopropylmethyl)desosaminyl erythronolide A |
| | (Compound No. 2) |
| 3. | 3-O-(4-Fluorophenyl)acetyl-5-O-(3′-N-desmethyl-3′-N- |
| | cyclopropylmethyl)desosaminyl erythronolide A |
| | (Compound No. 3) |
| 4. | 3-O-(4-Fluorophenyl)acetyl-5-O-[3′-N-desmethyl-3′- |
| | N-(4-fluoro)benzyl]desosaminyl-6-O-methyl erythronolide |
| | A (Compound No. 4) |
| 5. | 3-O-(2-Fluorophenyl)acetyl-5-O-[3′-N-desmethyl-3′- |
| | N-(4-fluoro)benzyl]desosaminyl-6-O-methyl erythronolide |
| | A (Compound No. 5) |
| 6. | 3-O-(3-Fluorophenyl)acetyl-5-O-[3′-N-desmethyl-3′- |
| | N-(4-fluoro)benzyl]desosaminyl-6-O-methyl erythronolide |
| | A (Compound No. 6) |
| 7. | 3-O-(2-Fluorophenyl)acetyl-5-O-(3′-N-desmethyl-3′- |
| | N-cyclopropyl) desosaminyl-6-O-methyl erythronolide |
| | A (Compound No. 7) |
| 8. | 3.O-(3-Fluorophenyl)acetyl-5-O-(3′-N-desmethyl-3′- |
| | N-ethyl)desosaminyl-6-O-methyl erythronolide A |
| | (Compound No. 8) |
| 9. | 3-O-(2-Fluorophenyl)acetyl-5-O-(3′-N-desmethyl-3′- |
| | N-ethyl)desoaminyl-6-O-methyl erythronolide A |
| | (Compound No. 9) |
| 10. | 3-O-(4-Fluorophenyl)acetyl-5-O-(3′-N-desmethyl-3′- |
| | N-ethyl)desoaminyl-6-O-methyl erythronolide A |
| | (Compound No. 10) |
| 11. | 3-O-(2-Fluorophenyl)acetyl-5-O-(3′-N-desmethyl-3′- |
| | N-benzyl)desosaminyl-6-O-methyl erythronolide A |
| | (Compound No. 11) |
| 12. | 3-O-(3-Fluorophenyl)acetyl-5-O-(3′-N-desmethyl-3′- |
| | N-benzyl) desosaminyl-6-O-methyl erythronolide A |
| | (Compound No. 12) |
| 13. | 3-O-(4-Fluorophenyl)acetyl-5-O-(3′-N-desmethyl-3′- |
| | N-benzyl) desosaminyl-6-O-methyl erythronolide A |
| | (Compound No. 13) |
| 14. | 3-O-(4-Fluorophenyl)acetyl-5-O-[3′-N-desmethyl-3′- |
| | N-(3,4-difluoro)benzyl] desosaminyl-6-O-methyl |
| | erythronolide (Compound No. 14) |
| 15. | 3-O-(4-Fluorophenyl)acetyl-5-O-[3′-N-desmethyl-3′- |
| | N-cyclopropyl) desosaminyl-6-O-methyl erythronolide |
| | A (Compound No. 15) |
| 16. | 3-O-(3-Fluorophenyl)acetyl-5-O-[3′-N-desmethyl-3′- |
| | N-cyclopropyl)desosaminyl-6-O-methyl erythronolide |
| | A (Compound No. 16) |
| 17. | 3-O-(4-Fluorophenyl)acetyl-5-O-[3′-N-desmethyl-3′- |
| | N-(3-hydroxy) benzyl] desosaminyl-6-O-methyl |
| | erythronolide A (Compound No. 17) |
| 18. | 3-O-(4-Fluorophenyl)acetyl-5-O-(3′-N-desmethyl-3′- |
| | N-propargyl)desosaminyl-6-O-methyl erythronolide |
| | A (Compound No. 18) |
| 19. | 3-O-(3-Fluorophenyl)acetyl-5-O-(3′-N-desmethyl-3′- |
| | N-allyl)desosaminyl-6-O-methyl erythronolide A |
| | (Compound No. 19) |
| 20. | 3-O-(4-Fluorophenyl)acetyl-5-O-(3′-N-desmethyl-3′- |
| | N-allyl)desosaminyl-6-O-methyl erythronolide A |
| | (Compound No. 20) |
| 21. | 3-O-(2-Fluorophenyl)acetyl-5-O-(3′-N-desmethyl-3′- |
| | N-propargyl)desosaminyl-6-O-methyl erythronolide |
| | A (Compound No. 21) |
| 22. | 3-O-(3-Fluorophenyl)acetyl-5-O-(3′-N-desmethyl-3′- |
| | N-propargyl)desosaminyl-6-O-methyl erythronolide |
| | A (Compound No. 22) |
| 23. | 3-O-(2-Fluorophenyl)acetyl-5-O-(3′-N-desmethyl-3′- |
| | N-allyl)desosaminyl-6-O-methyl erythronolide A |
| | (Compound No. 23) |
| 24. | 3-O-(4-Fluorophenyl)acetyl-5-O-(3′-N-desmethyl-3′- |
| | N-(4-nitro) benzyl)desosaminyl-6-O-methyl erythronolide |
| | A (Compound No. 24) |
| 25. | 3-O-(2-Nitrophenyl)acetyl-5-O-(3′-N-desmethyl-3′-N- |
| | cyclopropylmethyl)desosaminyl-6-O-methyl erythronolide |
| | A (Compound No. 25) |
| 26. | 3-O-(4-Nitrophenyl)acetyl-5-O-(3′-N-desmethyl-3′- |
| | N-cyclopropylmethyl) desosaminyl-6-O-methyl |
| | erythronolide A (Compound No. 26) |
| 27. | 3-O-(3-Nitrophenyl)acetyl-5-O-(3′-N-desmethyl-3′- |
| | N-benzyl)desosaminyl-6-O-methyl erythronolide |
| | A (Compound No. 27) |
| 28. | 3-O-(2-Nitrophenyl)acetyl-5-O-(3′-N-desmethyl-3′- |
| | N-ethyl)desoaminyl-6-O-methyl erythronolide A |
| | (Compound No. 28) |
| 29. | 3-O-(3-Nitrophenyl)acetyl-5-O-(3′-N-desmethyl-3′- |
| | N-ethyl)desoaminyl-6-O-methyl erythronolide A |
| | (Compound No. 29) |
| 30. | 3-O-(4-Nitrophenyl)acetyl-5-O-(3′-N-desmethyl-3′- |
| | N-ethyl)desoaminyl-6-O-methyl erythronolide A |
| | (Compound No. 30) |
| 3i: | 3-O-(2-Nitrophenyl)acetyl-5-O-(3′-N-desmethyl-3′- |
| | N-allyl)desosaminyl-6-O-methyl erythronolide A |
| | (Compound No. 31) |
| 32. | 3-O-(4-Nitrophenyl)acetyl-5-O-(3′-N-desmethyl-3′- |
| | N-allyl)desosaminyl-6-O-methyl erythronolide A |
| | (Compound No. 32) |
| 33. | 3-O-(3-Nitrophenyl)acetyl-5-O-(3′-N-desmethyl-3′- |
| | N-allyl)desosaminyl-6-O-methyl erythronolide A |
| | (Compound No. 33) |
| 34. | 3-O-(2-Nitrophenyl) acetyl-5-O-(3′-N-desmethyl-3′- |
| | N-propargyl) desosaminyl-6-O-methyl erythronolide |
| | A (Compound No. 34) |
| 35. | 3-O-(3-Nitrophenyl)acetyl-5-O-(3′-N-desmethyl-3′- |
| | N-propargyl)desosaminyl-6-O-methyl erythronolide A |
| | (Compound No. 35) |
| 36. | 3-O-(2-Nitrophenyl)acetyl-5-O-(3′-N-desmethyl-3′- |
| | N-isopropyl)desosaminyl-6-O-methyl erythronolide A |
| | (Compound No. 36) |
| 37. | 3-O-(3-Nitrophenyl)acetyl-5-O-(3′-N-desmethyl-3′- |
| | N-cyclopropyl)desosaminyl-6-O-methyl erythronolide |
| | A (Compound No. 37) |
| 38. | 3-O-(4-Nitrophenyl)acetyl-5-O-(3′-N-desmethyl-3′- |
| | N-cyclopropyl)desosaminyl-6-O-methyl erythronolide |
| | A (Compound No. 38) |
| 39. | 3-O-(2-Pyridyl)acetyl-5-O-(3′-N-desmethyl-3′- |
| | N-cyclopropylmethyl) desosaminyl-6-O-methyl |
| | erythronolide A (Compound No. 39) |
| 40. | 3-O-(4-Pyridyl)acetyl-5-O-(3′-N-desmethyl-3′- |
| | N-cyclopropylmethyl) desosaminyl-6-O-methyl |
| | erythronolide A (Compound No. 40) |
| 41. | 3-O-(2-Pyridyl)acetyl-5-O-(3′-N-desmethyl-3′- |
| | N-ethyl}desoaminyl-6-O-methyl erythronolide |
| | A (Compound No. 41) |
| 42. | 3-O-(3-Pyridyl)acetyl-5-O-(3′-N-desmethyl-3′- |
| | N-benzyl) desosaminyl-6-O-methyl erythronolide |
| | A (Compound No. 42) |
| 43. | 3-O-(2-Pyridyl)acetyl-5-O-[3′-N-desmethyl-3′- |
| | N-benzyl]desosaminyl-6-O-methyl erythronolide |
| | A (Compound No. 43) |
| 44. | 3-O-(2-Pyridyl)acetyl-5-O-(3′-N-desmethyl-3′- |
| | N-allyl)desosaminyl-6-O-methyl erythronolide |
| | A (Compound No. 44) |
| 45. | 3-O-(3-Pyridyl)acetyl-5-O-(3′-N-desmethyl-3′- |
| | N-allyl)desosaminyl-6-O-methyl erythronolide |
| | A (Compound No. 45) |
| 46. | 3-O-(2-Pyridyl)acetyl-5-O-(3′-N-desmethyl-3′- |
| | N-propargyl)desosaminyl-6-O-methyl erythronolide |
| | A (Compound No. 46) |
| 47. | 3-O-(3-Pyridyl)acetyl-5-O-(3′-N-desmethyl-3′- |
| | N-propargyl)desosaminyl-6-O-methyl erythronolide |
| | A (Compound No. 47) |
| 48. | 3-O-(4-Pyridyl)acetyl-5-O-(3′-N-desmethyl-3′- |
| | N-propargyl)desosaminyl-6-O-methyl erythronolide |
| | A (Compound No. 48) |
| 49. | 3-O-(4-Pyridyl)acetyl-5-O-(3′-N-desmethyl-3′- |
| | N-cyclopropyl)desosaminyl-6-O-methyl erythronolide |
| | A (Compound No. 49) |
| 50. | 3-O-(2-Pyridyl)acetyl-5-O-(3′-N-desmethyl-3′- |
| | N-cyclopropylmethyl)desosaminyl-6-O-methyl |
| | erythronolide A (Compound No. 50) |
| 51. | 3-O-(4-Pyridyl)acetyl-5-O-(3′-N-desmethyl-3′- |
| | N-ethyl)desosaminyl-6-O-methyl erythronolide A |
| | (Compound No. 51) |
| 52. | 3-O<3-Pyridyl)acetyl-5-O-(3′-N-desmethyl-3′-N- |
| | ethyl)desosaminyl-6-O-methyl erythronolide A |
| | (Compound No. 52) |
| 53. | 3-O-(4-Pyridyl) acetyl-5-O-N-desmethyl-3′-N- |
| | cyclopropylmethyl) desosaminyl-6-O-methyl |
| | erythronolide A (Compound No. 53) |
| 54. | 3-O-(4-Pyridyl) acetyl-5-O-(3′-N-desmethyl-3′- |
| | N-allyl)desosaminyl-6-O-methyl erythronolide A |
| | (Compound No. 54) |
| 55. | 3-O-(2-Pyridyl)acetyl-5-O-(3′-N-desmethyl-3′-N- |
| | cyclopropyl)desosaminyl-6-O-methyl erythronolide |
| | A (Compound No. 55) |
| 56. | 3-O-(Phenyl)acetyl-5-O-[(3′-N-desmethyl-3′-N- |
| | cyclopropylmethyl]desoaminyl-6-O-methyl |
| | erythronolide A (Compound No. 56) |
| 57. | 3-O-(Phenyl)acetyl-5-O-(3′-N-desmethyl-3′-N-(4- |
| | fluoro)benzyl)desoaminyl-6-O-methyl erythronolide |
| | A (Compound No. 57) |
| 58. | 3-O-(Phenyl)acetyl-5-O-(3′-N-desmethyl-3′-N- |
| | benzyl)desoaminyl-6-O-methyl erythronolide A |
| | (Compound No. 58) |
| 59. | 3-O-(2-Thiophene)acetyl-5-O-(3′-N-desmethyl-3′- |
| | N-cyclopropylmethyl) desosaminyl-6-O-methyl |
| | erythronolide A (Compound No. 59) |
| 60. | 3-O-(2-Thiophene)acetyl-5-O-(3′-N-desmethyl-3′- |
| | N-ethyl)desoaminyl-6-O-methyl erythronolide A |
| | (Compound No. 60) |
| 61. | 3-O-(2-Thiophene)acetyl-5-O-(3′-N-desmethyl-3′- |
| | N-benzyl)desosaminyl-6-O-methyl erythronolide A |
| | (Compound No. 61) |
| 62. | 3-O-(2-Thiophene)acetyl-5-O-[3′-N-desmethyl-3′- |
| | N-(3-hydroxy) benzyl] desosaminyl-6-O-methyl |
| | erythronolide A (Compound No. 62) |
| 63. | 3-O-(4-Chlorophenyl)acetyl-5-O-(3′-N-desmethyl-3′- |
| | N-ethyl)desoaminyl-6-O-methyl erythronolide A |
| | (Compound No. 63) |
| 64. | 3-O-(4-Chlorophenyl)acetyl-5-O-(3′-N-desmethyl-3′- |
| | N-benzyl)desosaminyl-6-O-methyl erythronolide A |
| | (Compound No. 64) |
| 65. | 3-O-(4-chlorophenyl)acetyl-5-O-[3′-N-desmethyl-3′- |
| | N-(3-hydroxy) benzyl] desosaminyl-6-O-methyl |
| | erythronolide A (Compound No. 65) |
| 66. | 3-O-(2-Methylphenyl)acetyl-5-O-[3′-N-desmethyl-3′- |
| | N-(4-hydroxy) benzyl] desosaminyl-6-O-methyl |
| | erythronolide A (Compound No. 66) |
| 67. | 3-O-(2-Methylphenyl)acetyl-5-O-(3′-N-desmethyl-3′-N- |
| | benzyl) desosaminyl-6-O-methyl erythronolide A |
| | (Compound No. 67) |
| 68. | 3-O-(4-Methylphenyl)acetyl-5-O-(3′-N-desmethyl-3′-N- |
| | benzyl) desosaminyl-6-O-methyl erythronolide A |
| | (Compound No. 68) |
| 69. | 3-O-(4-Methoxyphenyl)acetyl-5-O-(3′-N-desmethyl-3′-N- |
| | cyclopropylmethyl) desosaminyl-6-O-methyl erythronolide |
| | A (Compound No. 69) |
| 70. | 3-O-(4-Methoxyphenyl)acetyl-5-O-[3′-N-desmethyl-3′- |
| | N-(3,4-difluoro) benzyl]desosaminyl-6-O-methyl |
| | erythronolide A (Compound No. 70) |
| 71. | 3-O-(1-Naphthyl)acetyl-5-O-[3′-N-desmethyl-3′- |
| | N-(4-hydroxy) benzyl] desosaminyl-6-O-methyl |
| | erythronolide A (Compound No. 71) |
| 72. | 3-O-(1-Naphthyl)acetyl-5-O-(3′-N-desmethyl-3′-N- |
| | benzyl) desosaminyl-6-O-methyl erythronolide A |
| | (Compound No. 72) |
| 73. | 3-O-(2-Naphthyl)acetyl-5-O-(3′-N-desmethyl-3′-N- |
| | benzyl) desosaminyl-6-O-methyl erythronolide A |
| | (Compound No. 73) |
| 74. | 3-O-(2,4-Difluorophenyl)acetyl-5-O-(3′-N-desmethyl- |
| | 3′-N-benzyl) desosaminyl-6-O-methyl erythronolide |
| | A (Compound No. 74) |
| 75. | 3-O-(2,4-Difluorophenyl)acetyl-5-O-[3′-N-desmethyl- |
| | 3′-N-(3,4-difluoro) benzyl]desosaminyl-6-O-methyl |
| | erythronolide A (Compound No. 75) |
| 76. | 3-O-(2-Bromophenyl)acetyl-5-O-[3′-N-desmethyl-3′- |
| | N-(4-hydroxy) benzyl] desosaminyl-6-O-methyl |
| | erythronolide A (Compound No. 76) |
| 77. | 3-O-(2-Bromophenyl)acetyl-5-O-(3′-N-desmethyl-3′-N- |
| | benzyl) desosaminyl-6-O-methyl erythronolide A |
| | (Compound No. 77) |
| 78. | 3-O-(3-Indole)acetyl-5-O-(3′-N-desmethyl-3′-N- |
| | ethyl)desoaminyl-6-O-methyl erythronolide A |
| | (Compound No. 78) |
| 79. | 3-O-(2-Napthyl)acetyl-5-O-(3′-N-desmethyl-3′-N- |
| | ethyl)desoaminyl-6-O-methyl erythronolide A |
| | (Compound No. 79) |

The compounds described herein can be used as antibacterial agents, effective against gram positive or gram negative and atypical pathogens. The compounds are, in particular, effective against Staphylococcus aureus, Streptococcus pneumoniae, Enterococcus faecalis, Escherichia coli, Pseudomonas aeruginosa, and Haemophilus influenzae.

Functional groups contained in compounds described herein can form organic or inorganic acid salts,.which are within the scope of sound medical judgment suitable for use in contact with the tissue of humans and lower animals without undue toxicity, irritation, allergic response and the like. Also, other amino groups (primary and secondary) can be present, and can also for organic or inorganic salts. The resulting salts are useful by themselves and in the therapeutic composition. These salts may be prepared by conventional techniques, such as suspending the compound in water and then adding one equivalent of the organic acid such as acetic acid, oxalic acid, maleic acid, tartaric acid, citric acid, succinic acid, malonic acid, adipic acid, ascorbic acid, camphorenic acid, nicotinic acid, butyric acid, lactic acid, or glucuronic acid, or inorganic acids such as hydrochloric acid, hydrobromic acid, phosphoric acid, sulphuric acid, nitric acid, boric acid or perchloric acid.

The neutral solution of the resulting salt is subjected to rotary evaporation under diminished pressure to the volume necessary to ensure precipitation of the salt upon cooling, which is then filtered and dried. The salts of the compounds described herein may also be prepared under strictly non-aqueous conditions. For example, dissolving free base in a suitable organic solvent, adding one equivalent of the desired acid to the same solvent and stirring the solution at 0-5° C., causes the precipitation of the acid addition salt, which is then filtered, washed the solvent, and dried.

Alternatively, the solvent is stripped off completely to obtain the desired salt. These salts are often preferred for use in formulating the therapeutic compositions described herein, because they are crystalline and relatively recently more stable and water-soluble.

Making use of their antibacterial activity, the compounds disclosed herein may be administered to an animal for treatment orally, topically, rectally, internasally, or by parenteral route. The pharmaceutical compositions described herein comprise a pharmaceutically effective amount of a compound disclosed herein formulated together with one or more pharmaceutically acceptable carriers. The term “pharmaceutically acceptable carriers” is intended to include non-toxic, inert solid, semi-solid or liquid filler, diluent, encapsulating material or formulation auxiliary of any type.

Solid form preparations for oral administrations include capsules, tablet, pills, powder, granules, cachets and suppositories. For solid form preparations, the active compound is mixed with at least one inert, pharmaceutically acceptable excipient or carrier, such as sodium citrate, dicalcium phosphate and/or a filler or extenders such as starches, lactose, sucrose, glucose, mannitol or silicic acid; binders such as carboxymethylcellulose, alginates, gelatins, polyvinylpyrrolidinone, sucrose, or acacia; disintegrating agents such as agar-agar, calcium carbonate, potato starch, alginic acid, certain silicates or sodium carbonate; absorption accelerators such as quaternary ammonium compounds; wetting agents such as cetyl alcohol, glycerol mono stearate; adsorbants such as Kaolin; lubricants such as talc, calcium stearate, magnesium stearate, solid polyethyleneglycol, sodium lauryl sulphate or mixtures thereof. In the case of capsules, tablets, or pills, the dosage form may also comprise buffering agents.

The solid preparation of tablets, capsules, pills, or granules can be prepared with coating and shells such as enteric coating and other coatings well known in the pharmaceutical formulating art.

Liquid form preparations for oral administration can include pharmaceutically acceptable emulsions, solutions, suspensions, syrups and elixirs. For liquid form preparation, the active compound is mixed with water or other solvent, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (such as cottonseed, groundnut, corn, germ, olive, castor and sesame oil), glycerol, and fatty acid esters of sorbitan and mixture thereof. Besides inert diluents, the oral composition can also include adjutants, such as wetting agents, emulsifying agents, suspending agents, sweetening agents, flavoring agents and perfuming agents.

Injectable preparations such as sterile injections, aqueous or oleaginous suspensions may be formulated according to the art using suitable dispersing or wetting and suspending agent. Among the acceptable vehicles and solvents that may be employed are water, Ringer's solution, U. S. P. and isotonic sodium chloride.

Dosage forms for tropical or transdermal administration of a compound of the present invention include ointments, pastes, creams, lotions, gels, powders, solutions, sprays, inhalants or patches. The active compound is admixed under sterile condition with a pharmaceutically acceptable carrier and any needed preservatives or buffers as may be required. Ophthalmic formulations, ear drops, eye ointments, powder and solution are also contemplated as being within the scope of this invention.

Preferably, the pharmaceutical preparation is in unit dosage form. In such form, the preparation is subdivided into unit doses containing appropriate quantities of the active component. The unit dosage form can be packaged preparation, the package containing discrete capsules, powders, in vials or ampules, and ointments capsule, cachet, tablet, gel, cream itself or it can be the appropriate number of any of these packaged forms.

The quantity of active compound in unit dose of preparation may be varied or adjusted from less than 1 mg to several grams according to the particular application and potency of the active ingredient.

For therapeutic use, as agents for treating bacterial infections, the compounds disclosed here are utilized at initial dosages of about 3 mg to about 40 mg per kilogram daily. The dosages, however, may be varied depending upon the requirements of the patients and the compound being employed.

Pharmaceutically acceptable acid addition salts, solvates, enantiomers, diastereomers, polymorphs and metabolites having the same type of physiological or or therapeutic activity can also be used as therapeutic agents. Pharmaceutical compositions comprising the compounds disclosed herein, their enantiomers, diastereomers, pharmaceutically acceptable acid addition salts, pharmaceutically acceptable solvates, polymorphs, metabolites in combination with pharmaceutically acceptable carrier and optionally included excipients are also provided hereby.

In the above syntheses, where specific acids, bases, solvents, catalysts, reducing agents etc., are mentioned, it is to be understood that the other acids, bases, solvents, catalysts, reducing agents etc, may be used. Similarly, the reaction temperature and duration of the reaction may be adjusted according to the need.

The examples mentioned below demonstrate the general synthetic procedures as well as the specific preparation of particular compounds. The examples are provided to illustrate the details of the invention and should not be constrained to limit the scope of the present invention, which is defined by the claims appended hereto.

### Experimental Details

Various solvents, such as acetone, methanol, ethyl acetate, ether, tetrahydrofuran, pyridine, hexane and dichloromethane were dried using various drying agents according to procedures described in the literature. IR spectra were recorded as nujol mull or a thin neat film on a Perkin Elmer Paragone instrument, Nuclear Magnetic Resonance (NMR) were recorded on a Varion XL-300 MHz instrument using tetramethylsilane as an internal standard.

### EXAMPLE 1

### Preparation of 3-O-(Phenyl)acetyl-5-O-(3′-N-desmethyl-3′-N-cyclopropylmethyl)desosaminyl erythronolide A (Compound No. 56)

### Step 1: 5-O-Desosaminyl-6-O-methyl erythronolide A

To a solution of hydrochloric acid (˜1N, 160 mL) was added clarithromycin (10 g, 13.37 mmol) in portion at ambient temperature. After addition, the reaction mixture was stirred at 20° to 40° C. for about 40 minutes. The reaction mixture was neutralized with solid sodium bicarbonate (NaHCO₃) in cold and extracted with ethyl acetate. The organic layer was washed with water, brine, dried over anhydrous sodium sulphate (Na₂SO₄) and then the solvent was removed under vacuum to give white solid, 7.5 g (yield, 96%).

### Step 2: 3-Hydroxy-5-O-(2′-O-benzoyl)desosaminyl-6-O-methyl erythronolide A

5-O-Desosaminyl-6-O-methyl erythronolide A (7 g, 11.86 mmol, from step 1) was taken in dichloromethane (120 mL) and to it was added triethylamine (6.58 mL) and benzoic anhydride (6.7 g, 29.61 mmol) under argon atmosphere at 20° to 40° C. The reaction mixture was stirred at ambient temperature under argon atmosphere for about 48 hours. The reaction mixture was diluted with dichloromethane and the organic layer was washed successively with saturated sodium bicarbonate (NaHCO₃) solution and brine before drying over anhydrous sodium sulphate (Na₂SO₄). Removal of solvent afforded a white foamy solid, which was purified by column chromatography using 2% methanol in dichloromethane as eluent to obtain the desired product, 5.6 g (yield, 68%).

### Step 3: 3-O-(Phenyl)acetyl-5-O-(2′-O-benzoyl)desosaminyl-6-O-methyl erythronolide A

3-Hydroxy-5-O-(2′-O-benzoyl)desosaminyl-6-O-methyl erythronolide A (1 g, 1.44 mmol, from step 2) was dissolved in dry dichloromethane (30 mL), to this solution was added phenyl acetic acid (588 mg, 4.32 mmol), 1-ethyl-3-(3-dimethylaminopropyl) carbodiimide hydrochloride (40 mg, 2.1 mmol), 4-dimethylaminopyridine (256 mg, 2.1 mmol) under ice cooling condition. The whole reaction mixture was stirred at O° C. to ambient temperature for about 6 hours. This was poured into 5% sodium bicarbonate (NaHCO₃) solution and extracted with dichloromethane Organic layer was washed with water, dried over anhydrous sodium sulphate (Na₂SO₄) and the solvent was removed under vacuum. The crude product was then purified by column chromatography using hexane:acetone:triethylamine (9:1:0.1) as eluent to obtain the desired product, 700 mg (yield, 60%).

The following compounds were prepared by using above-mentioned sequence, using the appropriate acetic acid in step 3.
3-O-(3-Nitrophenyl)acetyl-5-O-(2′-O-benzoyl)desosaminyl-6-O-methyl erythronolide A
3-O-(4-Nitrophenyl)acetyl-5-O-(2′-O-benzoyl)desosaminyl-6-O-methyl erythronolide A
3-O-(2-Nitrophenyl)acetyl-5-O-(2′-O-benzoyl)desosaminyl-6-O-methyl erythronolide A
3-O-(2-Fluorophenyl)acetyl-5-O-(2′-O-benzoyl)-desosaminyl-6-O-methyl erythronolide A
3-O-(3-Fluorophenyl)acetyl-5-O-(2′-O-benzoyl)-desosaminyl-6-O-methyl erythronolide A
3-O-(4-Fluorophenyl)acetyl-5-O-(2′-O-benzoyl)-desosaminyl-6-O-methyl erythronolide A
3-O-(4-Chlorophenyl)acetyl-5-O-(2′-O-benzoyl)-desosaminyl-6-O-methyl erythronolide A
3-O-(2-Thiophene)acetyl-5-O-(2′-O-benzoyl)-desosaminyl-6-O-methyl erythronolide A
3-O-(4-Methoxyphenyl)acetyl-5-O-(2′-O-benzoyl)-desosaminyl-6-O-methyl erythronolide A
3-O-(4-Pyridyl)acetyl-5-O-(2′-O-benzoyl)-desosaminyl-6-O-methyl erythronolide A

### Step 4: 3-O-(Phenyl)acetyl-5-O-desosaminyl-6-O-methyl erythronolide A

3-O-(Phenyl)acetyl-5-O-(2′-O-benzoyl)desosaminyl-6-O-methyl erythronolide A (2.9 g, 3.57 mmol, from step 3) was taken in methanol (60 mL). The whole reaction mixture was heated at 60° C. for about 20 hours. Methanol was removed and the residue was purified by column chromatography using hexane: acetone: triethylamine (9:1:0.1) as eluent to obtain the desired product, 2.4 g (yield, 94%).

The following compounds were prepared analogously.
3-O-(4-Nitrophenyl)acetyl-5-O-desosaminyl-6-O-methyl erythronolide A
3-O-(3-Nitrophenyl)acetyl-5-O-desosaminyl-6-O-methyl erythronolide A
3-O-(2-Nitrophenyl)acetyl-5-O-desosaminyl-6-O-methyl erythronolide A
3-O-(2-Fluorophenyl)acetyl-5-O-desosaminyl-6-O-methyl erythronolide A
3-O-(3-Fluorophenyl)acetyl-5-O-desosaminyl-6-O-methyl erythronolide A
3-O-(4-Fluorophenyl)acetyl-5-O-desosaminyl-6-O-methyl erythronolide A
3-O-(4-Chlorophenyl)acetyl-5-O-desosaminyl-6-O-methyl erythronolide A
3-O-(4-Methoxyphenyl)acetyl-5-O-desosaminyl-6-O-methyl erythronolide A
3-O-(2-Thiophene)acetyl-5-O-desosaminyl-6-O-methyl erythronolide A
3-O-(4-Pyridyl)acetyl-5-O-desosaminyl-6-O-methyl erythronolide A

### Step 5: 3-O-(Phenyl)acetyl-5-O-(3′-N-desmethyl)desosaminyl-6-O-methyl erythronolide A

To a solution of 3-O-(Phenyl)acetyl-5-O-desosaminyl-6-O-methyl erythronolide A (500 mg, 0.71 mmol, from step 4) in dry acetonitrile (20 mL), was added N-iodosuccinimide (179 mg, 0.79 mmol) in portion. The whole reaction mixture was stirred for about 15-20 hours. To this reaction mixture was added sodium bisulphite (NaHSO₃) solution till the color become pale yellow. To this was added saturated sodium bicarbonate (NaHCO₃) solution. The whole reaction mixture was stirred for about 1 hour and then extracted with ethyl acetate. Organic layer was washed with water, dried over anhydrous sodium sulphate (Na₂SO₄) and the solvent was removed under vacuum. The crude product was then purified by column chromatography using hexane:acetone:TEA (1:1:0.1) to obtain the desired product, 450 mg (yield, 29%).

The following compounds were prepared analogously.
3-O-(2-Fluorophenyl)acetyl-5-O-(3′-N-desmethyl)desosaminyl-6-O-methyl erythronolide A
3-O-(3-Fluorophenyl)acetyl-5-O-(3′-N-desmethyl)desosaminyl-6-O-methyl erythronolide A
3-O-(4-Fluorophenyl)acetyl-5-O-(3′-N-desmethyl)desosaminyl-6-O-methyl erythronolide A
3-O-(4-Chlorophenyl)acetyl-5-O-(3′-N-desmethyl)desosaminyl-6-O-methyl erythronolide A
3-O-(4-Methoxyphenyl)acetyl-5-O-(3′-N-desmethyl)desosaminyl-6-O-methyl erythronolide A
3-O-(2-Thiophene)acetyl-5-O-(3′-N-desmethyl)desosaminyl-6-O-methyl erythronolide A
3-O-(2-Nitrophenyl)acetyl-5-O-(3′-N-desmethyl)desosaminyl-6-O-methyl erythronolide A
3-O-(3-Nitrophenyl)acetyl-5-O-(3′-N-desmethyl)desosaminyl-6-O-methyl erythronolide A
3-O-(4-Nitrophenyl)acetyl-5-O-(3′-N-desmethyl)desosaminyl-6-O-methyl erythronolide A
3-O-(2-Pyridyl)acetyl-5-O-(3′-N-desmethyl)desosaminyl-6-O-methyl erythronolide A
3-O-(4-Pyridyl)acetyl-5-O-(3′-N-desmethyl)desosaminyl-6-O-methyl erythronolide A

### Step 6: 3-O-(Phenyl)acetyl-5-O-(3′-N-desmethyl-3′-N-cyclopropylmethyl)desosaminyl-6-O-methyl erythronolide A

3-O-(phenyl)acetyl-5-O-(3′-N-desmethyl)desosaminyl erythronolide A (200 mg, 0.29 mmol, from step 5) was dissolved in dry acetonitrile (5 mL), to this was added anhydrous sodium bicarbonate (NaHCO₃, 120 mg, 1.43 mmol), followed by bromomethylcyclopropane (75 mg, 0.5 mmol). The whole reaction mixture was stirred at ambient temperature under argon atmosphere for about 60 hours. The reaction mixture was diluted with ethyl acetate and the organic layer was washed with water, dried over anhydrous sodium sulphate (Na₂SO₄) and the solvent was removed under vacuum. This crude (Na₂SO₄) product was then purified by column chromatography using hexane:acetone:triethylamine (95:5:1) as eluent to obtain the desired product, 120 mg (56%). m/z=766 (M+H)

The following compounds were prepared analogously.
3-O-(2-Fluorophenyl)acetyl-5-O-(3′-N-desmethyl-3′-N-cyclopropylmethyl)desosaminyl erythronolide A (Compound No. 2)
m/z=766 (M+H)
3-O-(4-Fluorophenyl)acetyl-5-O-(3′-N-desmethyl-3′-N-cyclopropylmethyl)desosaminyl erythronolide A (Compound No. 3)
m/z=766 (M+H)
3-O-(2-Nitrophenyl)acetyl-5-O-(3′-N-desmethyl-3′-N-cyclopropylmethyl)desosaminyl-6-O-methyl erythronolide A (Compound No. 25)
m/z=793 (M+H)
3-O-(4-Nitrophenyl)acetyl-5-O-(3′-N-desmethyl-3′-N-cyclopropylmethyl)desosaminyl-6-O-methyl erythronolide A (Compound No. 26)
m/z=793 (M+H)
3-O-(2-Thiophene)acetyl-5-O-(3′-N-desmethyl-3′-N-cyclopropylmethyl)desosaminyl-6-O-methyl erythronolide A (Compound No. 59)
m/z=754 (M+H)
3-O-(4-Methoxyphenyl)acetyl-5-O-(3′-N-desmethyl-3′-N-cyclopropylmethyl)desosaminyl-6-O-methyl erythronolide A (Compound No. 69)
m/z=778 (M+H)
3-O-(2-Pyridyl)acetyl-5-O-(3′-N-desmethyl-3′-N-cyclopropylmethyl)desosaminyl-6-O-methyl erythronolide A (Compound No. 50)
m/z=749 (M+H)
3-O-(4-Pyridyl)acetyl-5-O-(3′-N-desmethyl-3′-N-cyclopropyl)desosaminyl-6-O-methyl erythronolide A (Compound No. 49)
m/z=749 (M+H)
3-O-(2-Nitrophenyl)acetyl-5-O-(3′-N-desmethyl-3′-N-allyl)desosaminyl-6-O-methyl erythronolide A (Compound No. 31)
m/z=779 (M+H)
3-O-(3-Nitrophenyl)acetyl-5-O-(3′-N-desmethyl-3′-N-allyl)desosaminyl-6-O-methyl erythronolide A (Compound No. 33)
m/z=779 (M+H)
3-O-(4-Nitrophenyl)acetyl-5-O-(31-N-desmethyl-3′-N-allyl)desosaminyl-6-O-methyl erythronolide A (Compound No. 32)
m/z=779 (M+H)
3-O-(4-Pyridyl)acetyl-5-O-(3′-N-desmethyl-3′-N-cyclopropylmethyl)desosaminyl-6-O-methyl erythronolide A (Compound No. 40)
m/z=749 (M+H)

### EXAMPLE 2

### Preparation of 3-O-(4-Fluorophenyl)acetyl-5-O-[3′-N-desmethyl-3′-N-(4-fluoro)benzyl]desosaminyl-6-O-methyl erythronolide A (Compound No. 4)

3-O-(4-Fluorophenyl)acetyl-5-O-(3′-N-desmethyl)desosaminyl-6-O-methyl erythronolide A (350 mg, 0.49 mmol, example 1, step 5) was dissolved in methanol, to this was added 4-fluorobenzaldehyde (121 mg, 0.91 mmol) and glacial acetic acid (294 mg, 4.9 mmol). The whole reaction mixture was stirred at 0° C. for about 1.0 hours, then sodium cyanoborohydride (60 mg, 0.983 mmol) was added. The whole reaction mixture was stirred overnight. The reaction mixture was diluted with ethyl acetate followed by sodium bicarbonate (NaHCO₃) solution. Organic layer was washed with water, dried over anhydrous sodium sulphate and the solvent was removed under vacuum. This crude product was then purified by column chromatography using hexane:acetone:triethylamine (9:1:0.1) as eluent to obtain the desired product, 200 mg (yield, 50%).

m/z=820 (M+H)

The following compounds were prepared analogously using the appropriate aldehyde.
3-O-(2-Fluorophenyl)acetyl-5-O-[3′-N-desmethyl-3′-N-(4-fluoro)benzyl]desosaminyl-6-O-methyl erythronolide A (Compound No. 5)
m/z=820 (M+H)
3-O-(3-Fluorophenyl)acetyl-5-O-[3′-N-desmethyl-3′-N-(4-fluoro)benzyl]desosaminyl-6-O-methyl erythronolide A (Compound No. 6)
m/z=820 (M+H)
3-O-(2-Fluorophenyl)acetyl-5-O-(3′-N-desmethyl-3′-N-cyclopropyl)desosaminyl-6-O-methyl erythronolide A (Compound No. 7)
m/z=752 (M+H)

This title compound can be prepared using ethoxy trimethylsilyloxycyclopropyl instead of benzaldehyde.
3-O-(3-Nitrophenyl)acetyl-5-O-(3′-N-desmethyl-3′-N-benzyl)desosaminyl-6-O-methyl erythronolide A (Compound No. 27)
m/z=829 (M+H)
3-O-(Phenyl)acetyl-5-O-[3′-N-desmethyl-3′-N-(4-fluoro)benzyl]desosaminyl-6-O-methyl erythronolide A (Compound No. 57)
m/z=802 (M+H)

### EXAMPLE 3

### Preparation of 3-O-(3-Fluorophenyl)acetyl-5-O-(3′-N-desmethyl-3′-N-ethyl)desosaminyl-6-O-methyl erythronolide A (Compound No. 8)

### Step 1: 3′-N-Desmethyl Clarithromycin

To a solution of clarithromycin (10 g, 13.35 mmol) in dry acetonitrile (200 mL) was added N-iodosuccinimide (4.2 g, 18.66 mmol) in portion. The whole reaction mixture was stirred at ambient temperature for about 24 hours. To this reaction mixture was added sodium bisulphite (NaHSO₃) and then sodium carbonate (Na₂CO₃) solution. The whole reaction mixture was then stirred for about 3 hours and extracted with ethyl acetate, dried over anhydrous sodium sulphate (Na₂SO₄) and the solvent was removed under vacuum. The crude product was purified by column chromatography using hexane:acetone:triethylamine (6:1:0.1) as eluent to obtain the desired product, 9.3 g (yield, 47.4%).

### Step 2: 3′-N-Desmethyl-3′-N-ethyl clarithromycin

Method A: To a solution of 3′-N-desmethyl clarithromycin (2 g, 2.7 mmol, from step 1) in dry dimethyformamide (DMF, 15 mL) was added anhydrous potassium carbonate (K₂CO₃, 1.80 g, 13.08 mmol). The whole reaction mixture was stirred for about 5 minutes. Then ethyl iodide (0.61 g, 3.91 mmol) was added. The whole reaction mixture was stirred at ambient temperature for about 14-16 hours. It was filtered through the celite bed. Dimethyformamide (DMF) was removed under vacuum. The residue was dissolved in ethyl acetate and washed with water. The organic layer was dried over sodium sulphate (Na₂SO₄) and the solvent was removed under vacuum. The crude product was then purified by column chromatography using hexane:acetone:triethylamine (9:1:0.1) as eluent to obtain the desired product, 1 g (yield, 48%).

Method B: To a solution of 3′-N-desmethyl clarithromycin (2 g, 2.72 mmol, from step 1) in acetonitrile (30 mL) was added ethyl iodide (4.26 g, 27.32 mmol) and diisopropylethylamine (3.52 g, 27.32 mmol). The reaction mixture refluxed for about 4-5 hours. The reaction mixture was diluted with the dichloromethane and washed with 5% sodium bicarbonate (NaHCO₃) solution. The organic layer was dried over anhydrous sodium sulphate (Na₂SO₄) and the solvent was removed under vacuum. It was purified by column chromatography using hexane:acetone:triethylamine (9:1:0.1) to obtain the desired product, 1.1 g (yield, 53%).

### Step 3: 3-Hydroxy-5-O-(3′-N-desmethyl-3′-N-ethyl)desosaminyl-6-O-methyl erythronolide A

3′-N-Desmethyl-3′-N-ethyl-6-O-methyl erythronolide A (4.5 g, 5.89 mmol, from step 2) was added to a cold solution of hydrochloride (˜1N, 60 mL). It was stirred for about 1 hour, neutralized with solid sodium bicarbonate (NaHCO₃) and extracted with ethyl acetate, dried over sodium sulphate (Na₂SO₄) and concentrated. The crude product was purified by column chromatography using 20% acetone in hexane to obtain the desired product, 2.9 g (yield, 81%).

### Step 4: 3-Hydroxy-5-O-(2′-benzoyl-3′-N-desmethyl-3′-N-ethyl)desosaminyl-6-O-methyl erythronolide A

3-Hydroxy-5-O-(3′-N-desmethyl-3′-N-ethyl)desosaminyl-6-O-methyl erythronolide A (2.9 g, 4.80 mmol, from step 3) was dissolved in dry dichloromethane. To this was added benzoic anhydride (2.15 g, 9.60 mmol) and triethylamine (2.2 g, 22 mmol). The whole reaction mixture was stirred at ambient temperature for about 12 hours. More benzoic anhydride (1.07 g, 2.4 mmol) was added, and reaction mixture was stirred for an additional 6 hours. The organic layer was washed with sodium bicarbonate (NaHCO₃) solution and then with water, dried over anhydrous sodium sulphate (Na₂SO₄) and the solvent was removed under vacuum to obtain crude product which on purification by column chromatography using 10% acetone in hexane gave the desired product, 1.8 g (yield, 53%).

### Step 5: 3-O-(3-Fluorophenyl)acetyl-5-O-[(2′-O-benzoyl)-3′-N-desmethyl-3′-N-ethyl]desoaminyl-6-O-methyl erythronolide A

To a cold solution of 3-hydroxy-5-O-[(2′-benzoyl)-3′-N-desmethyl-3′-N-ethyl]desosaminyl-6-O-methyl erythronolide A (400 mg, 0.56 mmol, from step 4) was added DCC (230 mg, 1.30 mmol), DMAP (139 mg, 1.31 mmol), pyridine (223 mg, 2.8 mmol) and 3-fluorophenyl acetic acid (174 mg, 1.31 mmol). The reaction mixture was stirred at 0° to 20° C. for about 12 hours. Another equivalent each of 3-fluorophenyl acetic acid and DCC were added. The reaction mixture was stirred for about 6 hours more, filtered through celite bed, washed sequentially with sodium bicarbonate (NaHCO₃) solution, dilute hydrochloride and finally with water. The organic layer was dried over anhydrous sodium sulphate (Na₂SO₄), and the solvent was removed under vacuum. The crude product was purified by column chromatography using hexane:acetone:triethylamine (9:1:0.1) to obtain the desired product, 400 mg (yield, 83.8%).

The following compounds were prepared analogously, using the appropriate acetic acid.
3-O-(2-Fluorophenyl)acetyl-5-O-[(2′-O-benzoyl)-3′-N-desmethyl-3′-N-ethyl]desoaminyl-6-O-methyl erythronolide A
3-O-(4-Fluorophenyl)acetyl-5-O-[(2′-O-benzoyl)-3′-N-desmethyl-3′-N-ethyl]desoaminyl-6-O-methyl erythronolide A
3-O-(2-Nitrophenyl)acetyl-5-O-[(2′-O-benzoyl)-3′-N-desmethyl-3′-N-ethyl]desoaminyl-6-O-methyl erythronolide A
3-O-(3-Nitrophenyl)acetyl-5-O-[(2′-O-benzoyl)-3′-N-desmethyl-3′-N-ethyl]desoaminyl-6-O-methyl erythronolide A
3-O-(4-Nitrophenyl)acetyl-5-O-[(2′-O-benzoyl)-3′-N-desmethyl-3′-N-ethyl]desoaminyl-6-O-methyl erythronolide A
3-O-(2-Pyridyl)acetyl-5-O-[(2′-O-benzoyl)-3′-N-desmethyl-3′-N-ethyl}desoaminyl-6-O-methyl erythronolide A
3-O-Phenyl)acetyl-5-O-[(2′-O-benzoyl)-3′-N-desmethyl-3′-N-ethyl]desoaminyl-6-O-methyl erythronolide A
3-O-(3-Indole)acetyl-5-O-[(2′-O-benzoyl)-3′-N-desmethyl-3′-N-ethyl]desoaminyl-6-O-methyl erythronolide A
3-O-(2-Napthalene)acetyl-5-O-[(2′-O-benzoyl)-3′-N-desmethyl-3′-N-ethyl]desoaminyl-6-O-methyl erythronolide A
3-O-(2-Thiophene)acetyl-5-O-[(2′-O-benzoyl)-3′-N-desmethyl-3′-N-ethyl]desoaminyl-6-O-methyl erythronolide A
3-O-(4-Chlorophenyl)acetyl-5-O-[(2′-O-benzoyl)-3′-N-desmethyl-3′-N-ethyl]desoaminyl-6-O-methyl erythronolide A

### Step 6: 3-O-(3-Fluorophenyl)acetyl-5-O-(3′-N-desmethyl-3′-N-ethyl)desoaminyl-6-O-methyl erythronolide A

3-O-(3-Fluorophenyl)acetyl-5-O-[(2′-O-benzoyl)-3′-N-desmethyl-3′-N-ethyl]desoaminyl-6-O-methyl erythronolide A (350 mg, 0.42 mmol) was taken in methanol (60 mL) and refluxed for about 16 hours. Methanol was removed to get a gummy solid. This was purified by column chromatography using 2% methanol in chloroform as eluent to obtain the desired product, 0.29 g (94.7%).

m/z=740 (M+H)

The following compounds were prepared analogously.
3-O-(3-Pyridyl)acetyl-5-O-(3′-N-desmethyl-3′-N-ethyl)desosaminyl-6-O-methyl erythronolide A (Compound No. 52) m/z=723 (M+H)
3-O-(4-Pyridyl)acetyl-5-O-(3′-N-desmethyl-3′-N-ethyl)desosaminyl-6-O-methyl erythronolide A (Compound No. 51) m/z=723 (M+H)
3-O-(2-Fluorophenyl)acetyl-5-O-(3′-N-desmethyl-3′-N-ethyl)desoaminyl-6-O-methyl erythronolide A (Compound No. 9) m/z=740 (M+H)
3-O-(4-Fluorophenyl)acetyl-5-O-(3′-N-desmethyl-3′-N-ethyl)desoaminyl-6-O-methyl erythronolide A (Compound No. 10) m/z=740 (M+H)
3-O-(2-Nitrophenyl)acetyl-5-O-(3′-N-desmethyl-3′-N-ethyl)desoaminyl-6-O-methyl erythronolide A (Compound No. 28) m/z=767 (M+H)
3-O-(3-Nitrophenyl)acetyl-5-O-(3′-N-desmethyl-3′-N-ethyl)desoaminyl-6-O-methyl erythronolide A (Compound No. 29) m/z=767 (M+H)
3-O-(4-Nitrophenyl)acetyl-5-O-(3′-N-desmethyl-3′-N-ethyl)desoaminyl-6-O-methyl erythronolide A (Compound No. 30) m/z=767 (M+H)
3-O-(2-Pyridyl)acetyl-5-O-(3′-N-desmethyl-3′-N-ethyl)desoaminyl-6-O-methyl erythronolide A (Compound No. 41) m/z=723 (M+H)
3-O-(Phenyl)acetyl-5-O-(3′-N-desmethyl-3′-N-ethyl)desoaminyl-6-O-methyl erythronolide A m/z=761 (M+H) (Compound No. 80)
3-O-(3-Indole)acetyl-5-O-(3′-N-desmethyl-3′-N-ethyl)desoaminyl-6-O-methyl erythronolide A (Compound No. 78) m/z=761 (M+H)
3-O-(2-Napthyl)acetyl-5-O-(3′-N-desmethyl-3′-N-ethyl)desoaminyl-6-O-methyl erythronolide A m/z=772 (M+H) (Compound No. 79)
3-O-(2-Thiophene)acetyl-5-O-(3′-N-desmethyl-3′-N-ethyl)desoaminyl-6-O-methyl erythronolide A m/z=728 (M+H) (Compound No. 60)
3-O-(4-Chlorophenyl)acetyl-5-O-(3′-N-desmethyl-3′-N-ethyl)desoaminyl-6-O-methyl erythronolide A m/z=762 (M+H) (Compound No. 63)

### EXAMPLE 4

### Preparation of 3-O-(Phenyl)acetyl-5-O-(3′-N-desmethyl-3′-N-benzyl)desoaminyl-6-O-methyl erythronolide A (Compound No. 58)

### Step 1: 3-Hydroxy-5-O-desosaminyl-6-O-methyl erythronolide A

To a solution of hydrochloride (˜1N, 160 mL) was added clarithromycin (10 g, 13.37 mmol) in portion over a period of about 15 minutes. The reaction mixture was neutralized with solid sodium bicarbonate (NaHCO₃) in cold and extracted with ethyl acetate. The organic layer was washed with water, brine, dried over anhydrous sodium sulphate (Na₂SO₄) and the solvent was removed to give a crude product, 7.2 g (yield, 91%).

### Step 2: 3-Hydroxy-5-O-(2′-O-benzoyl)desosaminyl-6-O-methyl erythronolide A

A solution of 3-hydroxy-5-O-desosaminyl-6-O-methyl erythronolide A (15 g, 25.4 mmol), benzoic anhydride (14.4 g, 64 mmol), triethylamine (12.8 g, 127 mmol) in dry dichloromethane (150 mL) was stirred at 30° C. for a period of about 48 hours. The organic matter was extracted with dichloromethane, washed successively with sodium bicarbonate (NaHCO₃), water, brine, and dried over sodium sulphate (Na₂SO₄) and the solvent was removed under vacuum to get crude residue. The crude product was purified by column chromatography over a SiO₂ bed thoroughly neutralized with triethyl amine (gradient elution with a 10-20% acetone in hexanes), 10.4 g (yield, 59%).

### Step 3: 3-O-(Phenyl)acetyl-5-O-(2′-O-benzoyl)desosaminyl-6-O-methyl erythronolide A

To a solution of 3-hydroxy-5-O-(2′-benzoyl)desosaminyl-6-O-methyl erythronolide A (2.0 g, 2.9 mmol), DCC (1.45 g, 7.2 mmol), DMAP (0.9 g, 7.2 mmol), pyridine (1.1 g, 14.4 mmol) in dry dichloromethane (30 mL) was added phenyl acetic acid (0.98 g, 7.2 mmol) at O° C. and the reaction mixture was allowed to attain 30° C. and stirred at that temperature for a period about 8-15 hours. The solid residue was removed by filteration over a celite bed. The organic matter was extracted with dichloromethane, washed successively with sodium bicarbonate (NaHCO₃), water, brine, and dried over anhydrous sodium sulphate (Na₂SO₄) and the solvent was removed under vacuum to obtain crude residue. The pure product was obtained by column chromatography over a SiO₂ bed thoroughly neutralized with triethylamine (gradient elution with a 10-20% acetone in hexanes), 1.7 g (74%).

### Step 4: 3-O-(Phenyl)acetyl-5-O-(2′-O-benzoyl-3′-N-desmethyl)desosaminyl-6-O-methyl erythronolide A

To a solution of 3-O-(phenyl)acetyl-5-O-(2′-benzoyl)desosaminyl-6-O-methyl erythronolide A (1.7 g, 2.1 mmol) in dry acetonitrile (80 mL) was added N-iodosuccinimide (0.61 g, 2.73 mmol) at O° C. and the reaction mixture was allowed to attain 30° C. and stirred at that temperature for a period of more than 24 hours. The reaction mixture was stirred with a 5% solution of sodium bisulphite followed by stirring with 5% sodium carbonate solution. The organic matter was extracted with ethyl acetate, washed successively with water, brine, and dried over anhydrous sodium sulphate (Na_{2—}SO₄) and the solvent was removed under vacuum to obtain crude residue. The pure product was obtained by column chromatography over a SiO₂ bed thoroughly neutralized with triethylamine (gradient elution with a 10-20% acetone in hexanes), 1.0 g (60%).

The following compounds were prepared analogously using the appropriate acetic acid in step 3.
3-O-(2-Thiophene)acetyl-5-O-(2′-O-benzoyl-3′-N-desmethyl)desosaminyl-6-O-methyl erythronolide A
3-O-(4-Chlorophenyl)acetyl-5-O-(2′-O-benzoyl-3′-N-desmethyl)desosaminyl-6-O-methyl erythronolide A
3-O-(2-Fluorophenyl)acetyl-5-O-(2′-O-benzoyl-3′-N-desmethyl)desosaminyl-6-O-methyl erythronolide A
3-O-(4-Chlorophenyl)acetyl-5-O-(2-O-benzoyl-3′-N-desmethyl)desosaminyl-6-O-methyl erythronolide A
3-O-(3-Fluorophenyl)acetyl-5-O-(2′-O-benzoyl-3′-N-desmethyl)desosaminyl-6-O-methyl erythronolide A
3-O-(3-Pyridyl)acetyl-5-O-(2′-O-benzoyl-3′-N-desmethyl)desosaminyl-6-O-methyl erythronolide A
3-O-(4-Fluorophenyl)acetyl-5-O-(2′-O-benzoyl-3′-N-desmethyl)desosaminyl-6-O-methyl erythronolide A
3-O-(2-Naphthyl)acetyl-5-O-(2′-O-benzoyl-3′-N-desmethyl)desosaminyl-6-O-methyl erythronolide A
3-O-(4-Methoxyphenyl)acetyl-5-O-(2′-O-benzoyl-3′-N-desmethyl)desosaminyl-6-O-methyl erythronolide A
3-O-(2,4-Difluorophenyl)acetyl-5-O-(2′-O-benzoyl-3′-N-desmethyl)desosaminyl-6-O-methyl erythronolide A
3-O-(2,4-Difluorophenyl)acetyl-5-O-(2′-O-benzoyl-3′-N-desmethyl)desosaminyl-6-O-methyl erythronolide A
3-O-(2-Bromophenyl)acetyl-5-O-(2′-O-benzoyl-3′-N-desmethyl)desosaminyl-6-O-methyl erythronolide A
3-O-(2-Bromophenyl)acetyl-5-O-(2′-O-benzoyl-3v-N-desmethyl)desosaminyl-6-O-methyl erythronolide A
3-O-(1-Naphthyl)acetyl-5-O-(2′-O-benzoyl-3′-N-desmethyl)desosaminyl-6-O-methyl erythronolide A
3-O-(1-Naphthyl)acetyl-5-O-(2′-O-benzoyl-3′-N-desmethyl)desosaminyl-6-O-methyl erythronolide A
3-O-(2-Methylphenyl)acetyl-5-O-(2′-O-benzoyl-3′-N-desmethyl)desosaminyl-6-O-methyl erythronolide A
3-O-(2-Methylphenyl)acetyl-5-O-(2′-O-benzoyl-3′-N-desmethyl)desosaminyl-6-O-methyl erythronolide A
3-O-(4-Methylphenyl)acetyl-5-O-(2′-O-benzoyl-3′-N-desmethyl)desosaminyl-6-O-methyl erythronolide A
3-O-(4-Fluorophenyl)acetyl-5-O-(2′-O-benzoyl-3′-N-desmethyl)desosaminyl-6-O-methyl erythronolide A
3-O-(4-Fluorophenyl)acetyl-5-O-(2′-O-benzoyl-3′-N-desmethyl)desosaminyl-6-O-methyl erythronolide A

### Step 5: 3-O-(Phenyl)acetyl-5-O-(3′-N-desmethyl)desosaminyl-6-O-methyl erythronolide A

A solution of 3-O-(phenyl)acetyl-5-O-(2′-O-benzoyl-3′-N-desmethyl)desosaminyl-6-O-methyl erythronolide A (0.5 g, 0.62 mmol) in methanol (30 mL) was heated at 75-80° C. for about 8 hours. Removal of the solvent under vacuum yielded a crude residue. Purification of the crude product over a bed of silica gel thoroughly neutralized with triethylamine (gradient elution with a 20-30% acetone in hexanes) afforded the desired product, 0.28 g (66%).

The following compounds were prepared analogously.
3-O-(Phenyl)acetyl-5-O-(3′-N-desmethyl)desosaminyl-6-O-methyl erythronolide A
3-O-(2-Thiophene)acetyl-5-O-(3′-N-desmethyl)desosaminyl-6-O-methyl erythronolide A
3-O-(4-Chlorophenyl)acetyl-5-O-(3′-N-desmethyl)desosaminyl-6-O-methyl erythronolide A
3-O-(2-Fluorophenyl)acetyl-5-O-(3′-N-desmethyl)desosaminyl-6-O-methyl erythronolide A
3-O-(4-Chlorophenyl)acetyl-5-O-(3′-N-desmethyl)desosaminyl-6-O-methyl erythronolide A
3-O-(3-Fluorophenyl)acetyl-5-O-(3′-N-desmethyl)desosaminyl-6-O-methyl erythronolide A
3-O-(3-Pyridyl)acetyl-5-O-(3′-N-desmethyl)desosaminyl-6-O-methyl erythronolide A
3-O-(4-Fluorophenyl)acetyl-5-O-(3′-N-desmethyl)desosaminyl-6-O-methyl erythronolide A
3-O-(2-Naphthyl)acetyl-5-O-(3′-N-desmethyl)desosaminyl-6-O-methyl erythronolide A
3-O-(4-Methoxyphenyl)acetyl-5-O-(3′-N-desmethyl)desosaminyl-6-O-methyl erythronolide A
3-O-(2,4-Difluorophenyl)acetyl-5-O-(3′-N-desmethyl)desosaminyl-6-O-methyl erythronolide A
3-O-(2,4-Difluorophenyl)acetyl-5-O-(3′-N-desmethyl)desosaminyl-6-O-methyl erythronolide A
3-O-(2-Bromophenyl)acetyl-5-O-(3′-N-desmethyl)desosaminyl-6-O-methyl erythronolide A
3-O-(2-Bromophenyl)acetyl-5-O-(3′-N-desmethyl)desosaminyl-6-O-methyl erythronolide A
3-O-(1-Naphthyl)acetyl-5-O-(3′-N-desmethyl)desosaminyl-6-O-methyl erythronolide A
3-O-(1-Naphthyl)acetyl-5-O-(3′-N-desmethyl)desosaminyl-6-O-methyl erythronolide A
3-O-(2-Methylphenyl)acetyl-5-O-(3′-N-desmethyl)desosaminyl-6-O-methyl erythronolide A
3-O-(2-Methylphenyl)acetyl-5-O-(3′-N-desmethyl)desosaminyl-6-O-methyl erythronolide A
3-O-(4-Methylphenyl)acetyl-5-O-(3′-N-desmethyl)desosaminyl-6-O-methyl erythronolide A
3-O-(4-Fluorophenyl)acetyl-5-O-(3′-N-desmethyl)desosaminyl-6-O-methyl erythronolide A

### Step 6: 3-O-(Phenyl)acetyl-5-O-(3′-N-desmethyl-3′-N-phenylmethyl)desosaminyl-6-O-methyl erythronolide A

To a solution of 3-O-(phenyl)acetyl-5-O-(3′-N-desmethyl)desosaminyl-6-O-methyl erythronolide A (0.5 g, 0.72 mmol) in methanol (10 mL) were added benzaldehyde (0.38 g, 3.6 mmol), acetic acid (0.43 g, 7.3 mmol), NaBH₃CN (0.23 g, 3.6 mmol) sequentially at O° C. and the reaction mixture was then allowed to attain room temperature, and stirred for a period of about 6-8 hours. Removal of the solvent under vacuum yielded a crude residue. Purification of the crude product over a bed of silica gel thoroughly neutralized with triethylamine (gradient elution with a 2-5% acetone in hexanes) afforded the title compound, 0.15 g (26.5%) m/z=784 (M+H)

The following compounds were prepared analogously, using the appropriate benzaldehyde or derivative.
3-O-(2-Thiophene)acetyl-5-O-(3′-N-desmethyl-3′-N-benzyl)desosaminyl-6-O-methyl erythronolide A (Compound No. 61) m/z=790 (M+H)
3-O-(4-Chlorophenyl)acetyl-5-O-(3′-N-desmethyl-3′-N-benzyl)desosaminyl-6-O-methyl erythronolide A (Compound No. 64) m/z=718 (M+H)
3-O-(2-Fluorophenyl)acetyl-5-O-(3′-N-desmethyl-3′-N-benzyl)desosaminyl-6-O-methyl erythronolide A (Compound No. 11) m/z=802 (M+H)
3-O-(4-Chlorophenyl)acetyl-5-O-[3′-N-desmethyl-3′-N-(3-hydroxy)phenylmethyl]desosaminyl-6-O-methyl erythronolide A (Compound no. 65) m/z=834 (M+H)
3-O-(2-Thiophene)acetyl-5-O-[3′-N-desmethyl-3′-N-(3-hydroxy)phenylmethyl]desosaminyl-6-O-methyl erythronolide A (Compound No. 62) m/z=806 (M+H)
3-O-(3-Fluorophenyl)acetyl-5-O-(3′-N-desmethyl-3′-N-benzyl)desosaminyl-6-O-methyl erythronolide A (Compound No. 12) m/z=802 (M+H)
3-O-(4-Fluorophenyl)acetyl-5-O-(3′-N-desmethyl-3′-N-benzyl)desosaminyl-6-O-methyl erythronolide A (Compound No. 13) m/z=802 (M+H)
3-O-(2-Naphthyl)acetyl-5-O-(3′-N-desmethyl-3′-N-benzyl)desosaminyl-6-O-methyl erythronolide A (Compound No. 73) m/z=834 (M+H)
3-O-(4-Methoxyphenyl)acetyl-5-O-[3′-N-desmethyl-3′-N-(3,4-difluoro)benzyl]desosaminyl-6-O-methyl erythronolide A (Compound No. 70) m/z=850 (M+H)
3-O-(2,4-Difluorophenyl)acetyl-5-O-(3′-N-desmethyl-3′-N-benzyl)desosaminyl-6-O-methyl erythronolide A (Compound No. 74) m/z=820 (M+H)
3-O-(2,4-Difluorophenyl)acetyl-5-O-[3′-N-desmethyl-3′-N-(3,4-difluoro)benzyl]desosaminyl-6-O-methyl erythronolide A (Compound No. 75) m/z=856 (M+H)
3-O-(2-Bromophenyl)acetyl-5-O-[3′-N-desmethyl-3′-N-(4-hydroxy)benzyl]desosaminyl-6-O-methyl erythronolide A (Compound No. 76) m/z=878 (N+H), 880 (M+2+H)
3-O-(2-Bromophenyl)acetyl-5-O-(3′-N-desmethyl-3′-N-benzyl)desosaminyl-6-O-methyl erythronolide A (Compound No. 77) m/z=862 (M+H), 864 (M+2+H)
3-O-(1-Naphthyl)acetyl-5-O-[3′-N-desmethyl-3′-N-(4-hydroxy)benzyl]desosaminyl-6-O-methyl erythronolide A (Compound No. 71) m/z=850 (M+H)
3-O-(1-Naphthyl)acetyl-5-O-(3′-N-desmethyl-3′-N-benzyl)desosaminyl-6-O-methyl erythronolide A (Compound No. 72) m/z=834 (M+H)
3-O-(2-Methylphenyl)acetyl-5-O-[3′-N-desmethyl-3′-N-(4-hydroxy)benzyl]desosaminyl-6-O-methyl erythronolide A (Compound No. 66) m/z=814 (M+H)
3-O-(2-Methylphenyl)acetyl-5-O-(3′-N-desmethyl-3′-N-benzyl)desosaminyl-6-O-methyl erythronolide A (Compound No. 67) m/z=798 (M+H)
3-O-(4-Methylphenyl)acetyl-5-O-(3′-N-desmethyl-3′-N-benzyl)desosaminyl-6-O-methyl erythronolide A (Compound No. 68)
3-O-(4-Fluorophenyl)acetyl-5-O-[3′-N-desmethyl-3′-N-(3,4-difluoro)benzyl]desosaminyl-6-O-methyl erythronolide A (Compound No. 14) m/z=838 (M+H)
3-O-(4-Fluorophenyl)acetyl-5-O-[3′-N-desmethyl-3′-N-(3-hydroxy)benzyl]desosaminyl-6-O-methyl erythronolide A (Compound No. 17) m/z=818 (M+H)
3-O-(4-Fluorophenyl)-5-O-(3′-N-desmethyl-3′-N-cyclopropyl)desosaminyl-6-O-methyl erythronolide A (Compound No. 15) m/z=752 (M+H)
3-O-(3-Fluorophenyl)acetyl-5-O-(3′-N-desmethyl-3′-N-cyclopropyl)desosaminyl-6-O-methyl erythronolide A (Compound No. 16) m/z=752 (M+H)
3-O-(2-Fluorophenyl)acetyl-5-O-(3′-N-desmethyl-3′-N-cyclopropyl)desosaminyl-6-O-methyl erythronolide A (Compound No. 7) m/z=752 (M+H)

### Method B: 3-O-(4-Fluorophenyl)acetyl-5-O-[3′-N-desmethyl-31-N-propargyl]desosaminyl-6-O-methyl erythronolide A (Compound No. 18)

To a solution of 3-O-(4-Fluorophenyl)acetyl-5-O-[3′-N-desmethyl]desosaminyl-6-O-methyl erythronolide A (300 mg, 0.42 mmol, from step 5) in acetonitrile (10 ml) was added sodium bicarbonate (177 mg, 2.11 mmol) and propargyl bromide (69 mg, 0.46 mmol, 80% in toluene) and the mixture was stirred under nitrogen at 35° C.-45° C. for 24 to 30 hours. The reaction mixture was diluted with ethyl acetate and the solution was washed with 5% sodium bicarbonate solution, brine, dried over anhydrous sodium sulphate (Na₂SO₄) and the solvent was removed under vacuum. The product was purified by column chromatography using 8-10% acetone in hexane to obtain the desired product, 60 mg (19%) m/z=750 (M+H).

The following compounds were prepared similarly:
3-O-(3-Fluorophenyl)acetyl-5-O-(3′-N-desmethyl-3′-N-propargyl)desosaminyl-6-O-methyl erythronolide A. (Compound No. 22) m/z=750 (M+H)
3-O-(2-Nitrophenyl)acetyl-5-O-(3′-N-desmethyl-3′-N-propargyl)desosaminyl-6-O-methyl erythronolide A. (Compound No. 34) m/z=750 (M+H)

### EXAMPLE 5

### Preparation of 3-O-(2-Fluorophenyl)acetyl-5-O-(3′-N-desmethyl-3′-N-propargyl)desosaminyl-6-O-methyl erythronolide A (Compound No. 21)

### Step 3: 3-Hydroxy-5-O-(2′-O-benzoyl-3′-N-desmethyl)desosaminyl-6-O-methyl erythronolide A

3-Hydroxy-5-O-(2′-O-benzoyl)desosaminyl-6-O-methyl erythronolide A (1.0 g, 1.44 mmol, example 1, step 2) was dissolved in dry acetonitrile (40 mL) and N-iodosuccinimide (422 mg, 1.87 mmol) was added at 0° C. The whole reaction mixture was stirred at ambient temperature for about 48 hours and then concentrated to dryness. To this residue was added methanol (4 mL) and saturated ammonium chloride solution (4 mL). The whole reaction mixture was heated at 80° C. for about 1 hour, pH of the solution was adjusted to 8. It was then extracted with ethyl acetate. Ethyl acetate layer was washed with water, brine, dried over anhydrous sodium sulphate (Na₂SO₄) and the solvent was removed under vacuum. The crude product was then purified by column chromatography using hexane:acetone:triethylamine (6:1:0.1) to obtain the desired product, 540 mg (55%).

### Step 4: 3-Hydroxy-5-O-(2′-O-benzoyl-3′-N-desmethyl-3′-N-propargyl)desosaminyl-6-O-methyl erythronolide A

To a solution of 3-hydroxy-5-O-(2′-O-benzoyl-3′-N-desmethyl)desosaminyl-6-O-methyl erythronolide A (1.0 g, 1.47 mmol, from step 3) in acetonitrile (30 mL) was added sodium bicarbonate (NaHCO₃, 515 mg, 6.13 mmol) and propargyl bromide (0.20 g, 1.68 mmol, 80% in toluene) and the mixture was stirred under nitrogen at 35° C. for about 14 hours. The reaction mixture was diluted with ethyl acetate and the solution was washed with 5% sodium bicarbonate (NaHCO₃) solution, brine, dried over anhydrous sodium sulphate (Na₂SO₄) and the solvent was removed under vacuum. The product was purified by column chromatography using 8-10% acetone in hexane to obtain the desired product, 650 mg (61.6%).

The following compounds were prepared analogously, using the appropriate bromide:
3-O-Hydroxy-5-O-(2′-O-benzoyl-3′-N-desmethyl-3′-N-allyl)desosaminyl-6-O-methyl erythronolide A
3-Hydroxy-5-O-(2′-O-benzoyl-3′-N-desmethyl-3′-N-cyclopropylmethyl)desosaminyl-6-O-methyl erythronolide A
3-Hydroxy-5-O-(2′-O-benzoyl-3′-N-desmethyl-3′-N-cyclopropyl)desosaminyl-6-O-methyl erythronolide A

### Step 5: 3-O-(2-Fluorophenyl)acetyl-5-O-(2′-O-benzoyl-3′-N-desmethyl-3′-N-propargyl)desosaminyl-6-O-methyl erythronolide A

To a cold solution of 3-hydroxy-5-O-(2′-O-benzoyl-3′-N-desmethyl-3′-N-propargyl)desosaminyl-6-O-methyl erythronolide A (2 g, 2.78 mmol, from step 4) in dichloromethane were added DCC (1.4 g, 6.96 mmol), DMAP (0.85 g, 6.96 mmol) pyridine (1.12 g, 14.17 mmol) and 2-fluorophenyl acetic acid (1.1 g, 6.96 mmol). The whole reaction mixture was stirred at O° C. to 40° C. for about 16 hours. It was filtered through celite bed and washed with ethyl acetate. Organic layer was washed with saturated sodium bicarbonate (NaHCO₃) solution, water, brine, dried over anhydrous sodium sulphate (Na₂SO₄) and the solvent was removed under vacuum. The crude product was then purified by column chromatography using 8-10% acetone in hexane to obtain the desired product, 1.8 g (7.5%).

The following compounds were prepared analogously, using the appropriate acetic acid.
3-O-(3-Fluorophenyl)acetyl-5-O-(2′-O-benzoyl-3′-N-desmethyl-3′-N-propargyl)desosaminyl-6-O-methyl erythronolide A
3-O-(4-Flurophenyl)acetyl-5-O-(2′-O-benzoyl-3′-N-desmethyl-3′-N-propargyl)desosaminyl-6-O-methyl erythronolide A
3-O-(3-Flurophenyl)acetyl-5-O-(2′-O-benzoyl-3′-N-desmethyl-3′-N-propargyl)desosaminyl-6-O-methyl erythronolide A
3-O-(4-Nitrophenyl)acetyl-5-O-(2′-O-benzoyl-3′-N-desmethyl-3′-N-propargyl)desosaminyl-6-O-methyl erythronolide A
3-O-(3-Nitrophenyl)acetyl-5-O-(2′-O-benzoyl-3′-N-desmethyl-3′-N-propargyl)desosaminyl-6-O-methyl erythronolide A
3-O-(4-Pyridyl)acetyl-5-O-(2′-O-benzoyl-3′-N-desmethyl-3′-N-allyl)desosaminyl-6-O-methyl erythronolide A

### Step 6: 3-O-(2-Flurophenyl)acetyl-5-O-(3′-N-desmethyl-3′-N-propargyl)desosaminyl-6-O-methyl erythronolide A (Method A)

3-O-(2-Flurophenyl)acetyl-5-O-(2′-O-benzoyl-3′-N-desmethyl-3′-N-propargyl)desosaminyl-6-O-methyl erythronolide A (300 mg, 0.36 mmol) was taken in methanol (10 mL) and heated with stirring for about 78 hours. Methanol was removed and the product was purified by column chromatography using 10% acetone in hexane to obtain the desired compound, 90 mg (48%). m/z=733 (M+H).

The following compounds were prepared similarly
3-O-(3-Fluorophenyl)acetyl-5-O-(2′-O-benzoyl-3′-N-desmethyl-3′-N-propargyl)desosaminyl-6-O-methyl erythronolide A
3-O-(4-Flurophenyl)acetyl-5-O-(2′-O-benzoyl-3′-N-desmethyl-3′-N-propargyl)desosaminyl-6-O-methyl erythronolide A
3-O-(3-Flurophenyl)acetyl-5-O-(2′-O-benzoyl-3′-N-desmethyl-3′-N-propargyl)desosaminyl-6-O-methyl erythronolide A
3-O-(4-Nitrophenyl)acetyl-5-O-(2′-O-benzoyl-3′-N-desmethyl-3′-N-propargyl)desosaminyl-6-O-methyl erythronolide A
3-O-(3-Nitrophenyl)acetyl-5-O-(2′-O-benzoyl-3′-N-desmethyl-3′-N-propargyl)desosaminyl-6-O-methyl erythronolide A
3-O-(4-Pyridyl)acetyl-5-O-(2′-O-benzoyl-3′-N-desmethyl-3′-N-allyl)desosaminyl-6-O-methyl erythronolide A

### Step 6:3-O-(2-Flurophenyl)acetyl-5-O-(3′-N-desmethyl-3′-N-propargyl)desosaminyl-6-O-methyl erythronolide A (Method A)

3-O-(2-Flurophenyl)acetyl-5-O-(2′-O-benzoyl-3′-N-desmethyl-3′-N-propargyl)desosaminyl-6-O-methyl erythronolide A (300 mg, 0.36 mmol) was taken in methanol (10 mL) and heated with stirring for about 78 hours. Methanol was removed and the product was purified by column chromatography using 10% acetone in hexane to obtain the desired compound, 90 mg (48%). m/z=733 (M+H)

The following compounds were prepared similarly
3-O-(4-Fluorophenyl)acetyl-5-O-(3′-N-desmethyl-3′-N-allyl)desosaminyl-6-O-methyl erythronolide A (Compound No. 20) m/z=752 (M+H)
3-O-(3-Nitrophenyl)acetyl-5-O-(3′-N-desmethyl-3′-N-propargyl)desosaminyl-6-O-methyl erythronolide A (Compound No. 35) m/z=777 (M+H)
3-O-(2-Nitrophenyl)acetyl-5-O-(3′-N-desmethyl-3′-N-isopropyl)desosaminyl-6-O-methyl erythronolide A (Compound No. 36) m/z=781 (M+H)
3-O-(2-Pyridyl)acetyl-5-O-(3′-N-desmethyl-3′-N-cyclopropylethyl)desosaminyl-6-O-methyl erythronolide A (Compound No. 39) m/z=749 (M+H)
3-O-(3-Pyridyl)acetyl-5-O-(3′-N-desmethyl-3′-N-benzyl)desosaminyl-6-O-methyl erythronolide A (Compound No. 42) m/z=785 (M+H)
3-O-(2-Pyridyl)acetyl-5-O-(3′-N-desmethyl-3′-N-benzyl)desosaminyl-6-O-methyl erythronolide A (Compound No. 43) m/z=785 (M+H)
3-O-(2-Pyridyl)acetyl-5-O-(3′-N-desmethyl-3′-N-allyl)desosaminyl-6-O-methyl erythronolide A (Compound No. 44) m/z=735 (M+H)
3-O-(3-Pyridyl)acetyl-5-O-(3′-N-desmethyl-3′-N-allyl)desosaminyl-6-O-methyl erythronolide A (Compound No. 45) m/z=735 (M+H)
3-O-(4-Pyridyl)acetyl-5-O-(3′-N-desmethyl-3′-N-propargyl)desosaminyl-6-O-methyl erythronolide A (Compound No. 48) m/z=733 (M+H)
3-O-(4-Pyridyl)acetyl-5-O-(3′-N-desmethyl-3′-N-cyclopropyl)desosaminyl-6-O-methyl erythronolide A (Compound No. 49) m/z=735 (M+H)
3-O-(4-Pyridyl)acetyl-5-O-(3′-N-desmethyl-3′-N-allyl)desosaminyl-6-O-methyl erythronolide A (Compound No. 54) m/z=735 (M+H)

### Method B: 3-O-(3-Fluorophenyl)acetyl-5-O-(3′-N-desmethyl-3′-N-allyl)desosaminyl-6-O-methyl erythronolide A (Compound No. 19)

3-O-(3-Fluorophenyl)acetyl-5-O-(2′-O-benzoyl-3′-N-desmethyl-3′-N-allyl)desosaminyl-6-O-methyl erythronolide A (1.6 g, 1.87 mmol) was taken in ethanol (130 ml) along with sodium bicarbonate (168 mg, 1.6 mmol) heated to 90° C. to 95° C. for 24 to 30 hours. Ethanol was removed and the product was purified by column chromatography to yield the desired product, 1.32 g (94.3%/). M/z=752 (M+H)

### Method C: 3-O-(2-Fluorophenyl)acetyl-5-O-(3′-N-Desmethyl-3′-N-allyl)desosaminyl-6-O-methyl erythronolide A (Compound No. 23)

3-O-(2-Fluorophenyl)acetyl-5-O-(3′-N-benzoyl-3′-N-allyl)desosaminyl-6-O-methyl erythronolide A (350 mg, 0.41 mmol) was taken in methanol (20 ml). To it was added sodium carbonate (86 mg, 0.82 mmol) and heated to 70° C. to 75° C. for 10 to 12 hours. Methanol was evaporated and the resulting compound was purified by column chromatography to yield the desired product, 100 mg (32.6%) (M+H)=752.

The following compounds were prepared similarly:
3-O-(2-Pyridyl)acetyl-5-O-(3′-N-desmethyl-3′-N-propargyl)desosaminyl-6-O-methyl erythronolide A (Compound No. 46) m/z=733 (M+H)
3-O-(3-Pyridyl)acetyl-5-O-(3′-N-desmethyl-3′-N-propargyl)desosaminyl-6-O-methyl erythronolide A (Compound No. 47) m/z=733 (M+H)
3-O-(3-Nitrophenyl)acetyl-5-O-(3′-N-desmethyl-3′-N-cyclopropyl)desosaminyl-6-O-methyl erythronolide A (Compound No. 37) m/z=779 (M+H)
3-O-(4-Nitrophenyl)acetyl-5-O-(3′-N-desmethyl-3′-N-cyclopropyl)desosaminyl-6-O-methyl erythronolide A (Compound No. 38) m/z=779 (M+H)

3-O-(2-Pyridyl)acetyl-5-O-(3′-N-desmethyl-3′-N-cyclopropyl)desosaminyl-6-O-methyl erythronolide A (Compound No. 55) m/z=735 (M+H)

TABLE 1

| | |
|---|---|
| | Formula I |
| 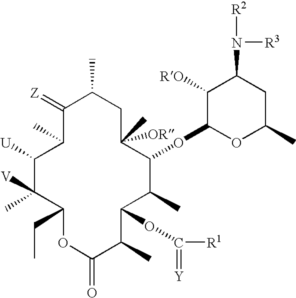 | |

(Formula I, wherein, R³═R″═CH₃, R′═H, U═V═OH, and Y═Z═O)

| Compound No. | R¹ | R² |
|---|---|---|
| 1 | 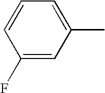 | 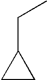 |
| 2 | 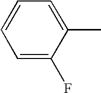 | 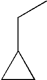 |
| 3 | 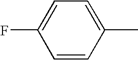 | 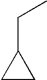 |
| 4 | 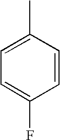 | 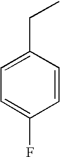 |
| 5 | 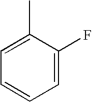 | 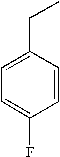 |
| 6 | 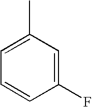 | 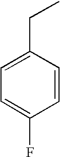 |
| 7 | 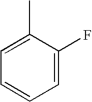 | 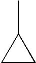 |
| 8 | 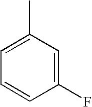 | C₂H₅ |
| 9 | 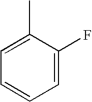 | C₂H₅ |
| 10 | 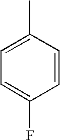 | C₂H₅ |
| 11 | 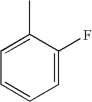 | 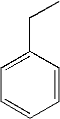 |
| 12 | 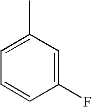 | 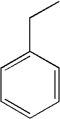 |
| 13 | 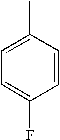 | 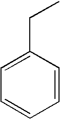 |
| 14 | 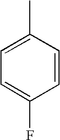 | 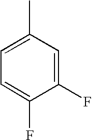 |
| 15 | 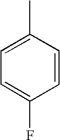 | 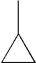 |
| 16 | 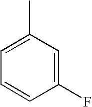 | 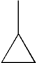 |
| 17 | 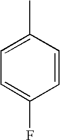 | 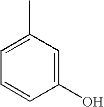 |
| 18 | 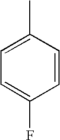 | 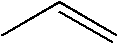 |
| 19 | 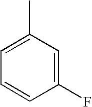 | 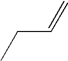 |
| 20 | 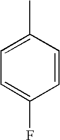 | 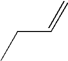 |
| 21 | 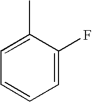 | 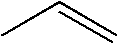 |
| 22 | 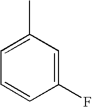 | 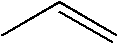 |
| 23 | 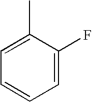 | 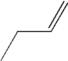 |
| 24 | 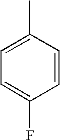 | 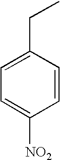 |
| 25 | 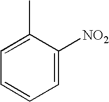 | 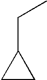 |
| 26 | 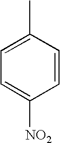 | 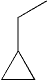 |
| 27 | 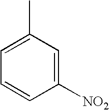 | 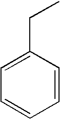 |
| 28 | 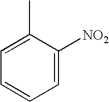 | C₂H₅ |
| 29 | 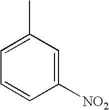 | C₂H₅ |
| 30 | 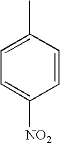 | C₂H₅ |
| 31 | 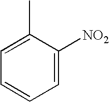 | 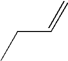 |
| 32 | 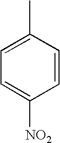 | 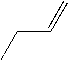 |
| 33 | 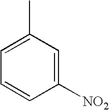 | 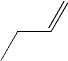 |
| 34 | 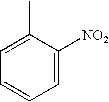 | 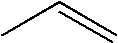 |
| 35 | 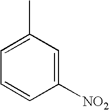 | 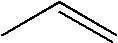 |
| 36 | 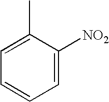 | 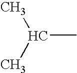 |
| 37 | 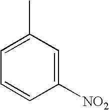 | 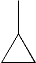 |
| 38 | 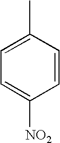 | 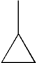 |
| 39 | 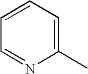 | 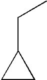 |
| 40 | 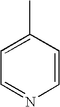 | 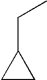 |
| 41 | 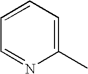 | C₂H₅ |
| 42 | 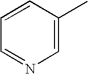 | 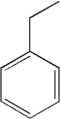 |
| 43 | 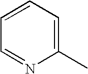 | 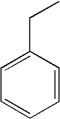 |
| 44 | 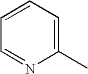 | 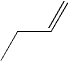 |
| 45 | 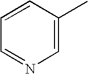 | 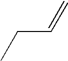 |
| 46 | 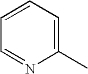 | 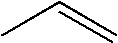 |
| 47 | 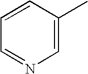 | 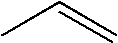 |
| 48 | 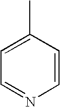 | 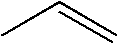 |
| 49 | 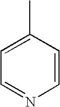 | 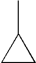 |
| 50 | 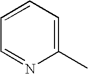 | 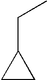 |
| 51 | 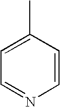 | C₂H₅ |
| 52 | 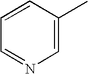 | C₂H₅ |
| 53 | 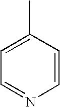 | 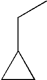 |
| 54 | 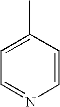 | 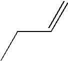 |
| 55 | 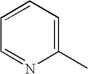 | 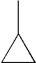 |
| 56 | 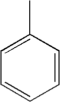 | 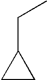 |
| 57 | 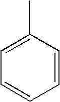 | 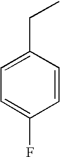 |
| 58 | 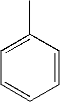 | 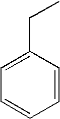 |
| 59 | 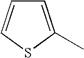 | 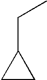 |
| 60 | 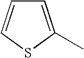 | C₂H₅ |
| 61 | 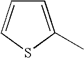 | 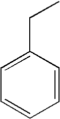 |
| 62 | 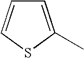 | 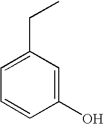 |
| 63 | 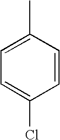 | C₂H₅ |
| 64 | 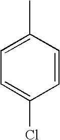 | 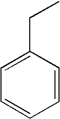 |
| 65 | 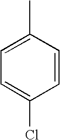 | 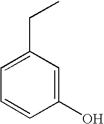 |
| 66 | 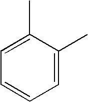 | 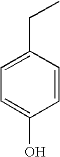 |
| 67 | 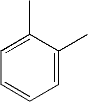 | 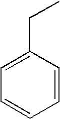 |
| 68 | 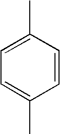 | 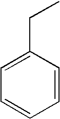 |
| 69 | 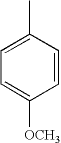 | 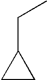 |
| 70 | 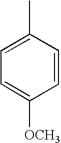 | 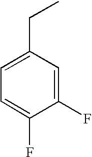 |
| 71 | 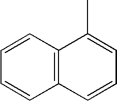 | 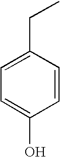 |
| 72 | 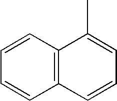 | 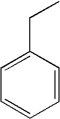 |
| 73 | 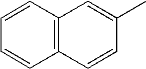 | 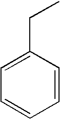 |
| 74 | 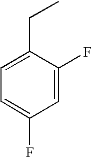 | 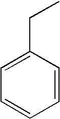 |
| 75 | 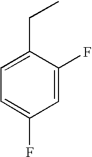 | 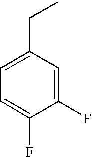 |
| 76 | 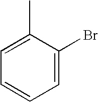 | 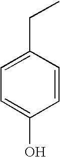 |
| 77 | 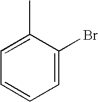 | 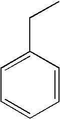 |
| 78 | 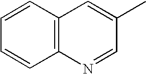 | C₂H₅ |
| 79 | 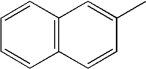 | C₂H₅ |
| 80 | 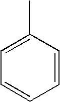 | C₂H₅ |

In Vitro Antibacterial Activity

Compounds of the invention displayed antibacterial activity when tested by the agar incorporation method (NCCLS agar dilution method). Minimum inhibitory concentrations (μg/ml) were obtained for representative compounds of the invention, which are given in Table 2.

Briefly, 1 mg/ml concentration of stock solutions of the compounds and erythromycin A, clarithromycin, telithromycin and clindamycin are prepared in dimethylsulfoxide. Drug dilutions and the agar plates are prepared by NCCLS method. Inoculum is prepared by direct colony suspensions in normal saline and adjusted to 0.5 McFarland turbidity and subsequently diluted as per NCCLS guidelines in order to obtain 10⁴ CFU/spot. CFU/ml of a few randomly selected cultures is performed. The cultures are replicated on the agar plates using replicator (Denley's multipoint). The plates are incubated at 35±2° C. and in 5% CO₂ atmosphere (for respiratory and Haemophilus strains) for 18-24 hours at 35±2° C. Minimum inhibitory concentrations (MIC) are determined after 18-24 hours. Q. C. strains are also included in each run of the study. The cation content of Mueller Hinton agar is checked by performing disk dilution of 10 μg gentamicin disk against Pseudomonas aeroginosa ATCC 27853. The zone of inhibition should fall between 16-21 mm. The concentration of drug at which there is complete disappearance of growth spot or formation of less than 10 colonies per spot is considered as MIC.

While the present invention has been described in terms of its specific embodiments, certain modifications and equivalents will be apparent to those skilled in the art and are intended to be included within the scope of the present invention.

TABLE 2
Minimum Inhibitory Concentrations for Particular Compounds Disclosed Herein

| | S. | S. | S. | S. | S. | S. | S. | S. | S. | S. | S. | | |
|---|---|---|---|---|---|---|---|---|---|---|---|---|---|
| | pneum | pneum | pneum | pneum | pneum | pneum | pneum | pneum | pneum | pneum | pneum | | H. inflenza |
| C.. | AB 14 | AB 29 | AB 30 | 1256 | 1275 | AB 34 | CS 1687 | 1251 | 1294 | ATCC | ATCC | MoraXella | ATCC |
| No. | (erm)* | (erm) | (erm) | (erm) | (erm) | (mef)** | (mef) | (mef) | (mef) | 49619 | 6303 | MI | 49247 |
| E | >16 | >16 | >16 | >16 | >16 | >16 | >16 | 8 | >16 | 0.008 | 0.03 | 0.06 | >16 |
| C | >16 | >16 | >16 | >16 | >16 | >16 | >16 | 8 | >16 | 0.008 | 0.008 | 0.125 | 8 |
| T | | 0.25 | 0.25 | 0.25 | 0.008 | 0.125 | 0.125 | 0.5 | 0.5 | 0.008 | 0.015 | 0.03 | 2 |
| Cl | >16 | | | | >16 | 0.03 | 0.06 | 0.03 | 0.03 | 0.015 | 0.06 | 2 | |
| 5 | >8 | | >8 | >8 | >16 | >8 | 2 | 2 | 2 | 2 | 2 | >8 | >128 |
| 16 | >16 | | >16 | 16 | >16 | >16 | 4 | 8 | 1 | 4 | 1 | >16 | >16 |
| 17 | >16 | <0.5 | >16 | >16 | >16 | >16 | 1 | 1 | 1 | <0.5 | <0.5 | 8 | >16 |
| 18 | 16 | 1 | >16 | >16 | >16 | 1 | 1 | 1 | 1 | 1 | 1 | 16 | >16 |
| 19 | >16 | 2 | >16 | >16 | >16 | 16 | 2 | 2 | 4 | 2 | 2 | 16 | |
| 20 | >16 | | >16 | 16 | >16 | >16 | 1 | 1 | 1 | 0.25 | 0.5 | 16 | >16 |
| 21 | >16 | >16 | >16 | >16 | >16 | >16 | 0.5 | 0.25 | 2 | >16 | 0.5 | 0.25 | >16 |
| 22 | >16 | | >16 | >16 | >16 | >16 | 1 | 4 | 4 | 0.5 | 0.5 | 4 | >16 |
| 23 | >16 | 1 | >16 | >16 | >16 | >16 | 2 | 2 | 2 | 2 | 2 | 16 | >16 |
| 24 | 8 | >16 | >16 | >16 | >16 | >16 | 0.5 | 1 | 2 | 0.5 | 0.5 | 2 | |
| 30 | >16 | <0.5 | >16 | >16 | >16 | >16 | 1 | 1 | 2 | 1 | 1 | 4 | >16 |
| 50 | >16 | 0.5 | >16 | >16 | >16 | >16 | 1 | 0.5 | 2 | 0.5 | 0.5 | 2 | >16 |
| 73 | >16 | >16 | >16 | >16 | >16 | >16 | 2 | 2 | >16 | 2 | 1 | 16 | |
| 74 | 16 | >16 | >16 | >16 | >16 | >16 | >16 | >16 | >16 | >16 | 8 | >16 | |

E: Erythromucin A, C: Clarithromycin, T: Telithromycin, Cl: Clindamycin, S. pneum: streptococcus pneumoniae
*Ribosomal modification (mutation) erm genes
**Efflux pump mef genes

We claim:

1. A compound having the structure of Formula I 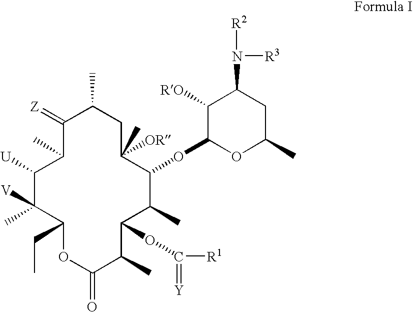
and its pharmaceutically acceptable acid addition salts, pharmaceutically acceptable solvates, enantiomers, diastereomers, polymorphs and metabolites, wherein
R¹ represents: lower alkyl (C₁-C₅) group, lower alkyl (C₁-C₅) having one or more halogen (F, Cl, Br, I) atoms as substituent (s), lower alkyl (C₁-C₅) amino group, lower alkyl amino (C₁-C₅) carbonyl group; lower alkoxy group (C₁-C₅); or five or six membered aryl or heteroaryl ring having 1 to 3 hetero atoms selected from the group consisting of oxygen, nitrogen, and sulphur, wherein the aryl or heteroaryl ring may be unsubstituted or substituted by 1 to 3 substituents independently selected from the group consisting of lower alkyl (C₁-C₅) group, lower alkyl (C₁-C₅) group having one or more halogen (F, Cl, Br, I) atoms, lower alkoxy (C₁-C₅) groups, lower alkyl (C₁-C₅) amino group, halogen atoms (F, Cl, Br, I), amino group, nitro group, hydroxy group, and cyano group;
R² and R³ are independently selected from: C₁-C₆ alkyl group optionally substituted with halogen atoms (F, Cl, Br, I); cycloalkyl (C₃-C₇) group; or five to six membered aryl or heteroaryl ring having 1 to 3 hetero atom independently selected from the group consisting of nitrogen, oxygen, and sulphur, wherein the aryl or heteroaryl ring may be unsubstituted or substituted by 1 to 3 substituents independently selected from the group consisting of lower alkyl (C₁-C₃), lower alkyl (C₁-C₃) group having one or more halogen (F, Cl, Br, I) atom as substituent(s), lower alkoxy (C₁-C₃) group, lower alkyl (C₁-C₃) amino group, halogen (F, Cl, Br, I) atoms, nitro group, hydroxy group, or cyano group; the above-mentioned C₁-C₆ alkyl group may be substituted by: NHCOR⁵, NHCOOR⁵, OCOR⁵, COR⁵ wherein R⁵ represents lower alkyl (C₁-C₅); five to six membered aryl or heteroaryl ring having 1 to 3 hetero atom independently selected from the group consisting of nitrogen, oxygen, and sulphur, wherein the aryl or heteroaryl ring may be unsubstituted or substituted by 1 to 3 substituents independently selected from the group consisting of lower alkyl (C₁-C₃), lower alkyl (C₁-C₃) group having one or more halogen (F, Cl, Br, I) atoms as substituent(s), lower alkoxy (C₁-C₃) group, lower alkyl (C₁-C₃) amino group, halogen (F, Cl, Br, I) atoms, nitro group, hydroxy group, and cyano group; C₂-C₆ alkenyl or alkyne group optionally substituted with halogen (F, Cl, Br, I) atoms or a group consisting of NHCOR⁵, NHCOOR⁵, COR⁵, OCOR⁵ (wherein R⁵ is as defined above); cycloalkyl (C₃-C₇) group; five or six membered aryl or heteroaryl ring having 1 to 3 hetero atom independently selected from the group consisting of nitrogen, oxygen, and sulphur, wherein the aryl or heteroaryl ring may be unsubstituted or substituted by 1 to 3 substituents independently selected from the group consisting of lower alkyl (C₁-C₃) group, lower alkyl (C₁-C₃) group having one or more halogen (F, Cl, Br, I) atoms as substituent(s), lower alkoxy (C₁-C₃) group, lower alkyl (C₁-C₃) amino, halogen (F, Cl, Br, I) atoms, nitro group, hydroxy group, amino group, and cyano group;
R′ represents hydrogen, or a hydroxy protecting group optionally selected from acetyl, benzoyl, butyldiphenylsilyl, methylthiomethyl, or methoxy methyl;
R″ represents hydrogen, or a lower alkyl (C₁-C₃) group;
Y represents oxygen or sulphur;
Z represents an oxygen atom or a group represented by NOR⁶, wherein R⁶ represents hydrogen atom, alkyl (C₁-C₆) group, alkyl (C₁-C₆) amino group, phenyl or benzyl group, or phenyl or benzyl group having 1 to 5 substituent independently selected from halogen (F, Cl, Br, I) atoms, lower alkyl (C₁-C₃) group, hydroxy group, nitro group, cyano group, or amino group;
U represents a hydroxy group: OR⁷, wherein R⁷ represents hydroxy protecting group selected from acetyl, benzoyl, butyldiphenylsilyl, methylthiomethyl, or methoxymethyl; or —NH(CH₂)ₙR⁸, wherein n represents 0 to 4 and R⁸ represents five or six membered aryl or heteroaryl ring having 1 to 4 hetero atom independently selected from the group consisting of nitrogen, oxygen, and sulphur, wherein the aryl or heteroaryl ring may be unsubstituted or substituted by one to three substituents independently selected from the group consisting of lower alkyl (C₁-C₃) group, lower alkyl (C₁-C₃) group having one or more halogen (F, Cl, Br, I) atoms as substituent (s), lower alkoxy (C₁-C₃) group, lower alkyl (C₁-C₃) amino, halogen (F, Cl, Br, I) atoms, nitro group, hydroxy group, amino group, and cyano group;
V represents: hydrogen atom; hydroxy group; or OR⁷, wherein R⁷ represents a hydroxy protecting group selected from the group consisting of acetyl, benzoyl, butyldiphenylsilyl, methylthiomethylm and methoxymethyl;
U and V may also together represent (with carbon atoms at the 1- and 12-positions on the erythronolide skeleton): a group represented by Formula 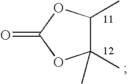
or a group represented by the Formula 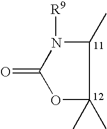
wherein R⁹ represents: hydrogen atom; alkyl (C₁-C₆) group, wherein the alkyl (C₁-C₆) may be unsubstituted or substituted by halogen (F, Cl, Br, I) atoms, five or six membered aryl or heteroaryl ring having 1 to 3 hetero atom independently selected from the group consisting of nitrogen, oxygen, and sulphur, wherein the aryl or heteroaryl ring may be unsubstituted or substituted by 1 to 3 substituents independently selected from the group consisting of lower alkyl (C₁-C₃) group, lower alkyl (C₁-C₃) group having one or more halogen (F, Cl, Br, I) atoms as substituent(s), lower alkoxy (C₁-C₃) group, lower alkyl (C₁-C₃) amino, halogen (F, Cl, Br, I) atoms, nitro group, hydroxy group, amino group, and cyano group.

2. A compound selected from the group consisting of:
3-O-(3-Fluorophenyl)acetyl-5-O-(3′-N-desmethyl-3′-N-cyclopropylmethyl)desosaminyl erythronolide A (Compound No. 1)
3-O-(2-Fluorophenyl)acetyl-5-O-(3′-N-desmethyl-3′-N-cyclopropylmethyl)desosaminyl erythronolide A (Compound No. 2)
3-O-(4-Fluorophenyl)acetyl-5-O-(3′-N-desmethyl-3′-N-cyclopropylmethyl)desosaminyl erythronolide A (Compound No. 3)
3-O-(4-Fluorophenyl)acetyl-5-O-[3′-N-desmethyl-3′-N-(4-fluoro)benzyl]desosaminyl-6-O-methyl erythronolide A (Compound No. 4)
3-O-(2-Fluorophenyl)acetyl-5-O-[3′-N-desmethyl-3′-N-(4-fluoro)benzyl]desosaminyl-6-O-methyl erythronolide A (Compound No. 5)
3-O-(3-Fluorophenyl)acetyl-5-O-[3′-N-desmethyl-3′-N-(4-fluoro)benzyl]desosaminyl-6-O-methyl erythronolide A (Compound No. 6)
3-O-(2-Fluorophenyl)acetyl-5-O-(3′-N-desmethyl-3′-N-cyclopropyl)desosaminyl-6-O-methyl erythronolide A (Compound No. 7)
3-O-(3-Fluorophenyl)acetyl-5-O-(3′-N-desmethyl-3′-N-ethyl)desosaminyl-6-O-methyl erythronolide A (Compound No. 8)
3-O-(2-Fluorophenyl)acetyl-5-O-(3′-N-desmethyl-3′-N-ethyl)desoaminyl-6-O-methyl erythronolide A (Compound No. 9)
3-O-(4-Fluorophenyl)acetyl-5-O-(3′-N-desmethyl-3′-N-ethyl)desoaminyl-6-O-methyl erythronolide A (Compound No. 10)
3-O-(2-Fluorophenyl)acetyl-5-O-(3′-N-desmethyl-3′-N-benzyl)desosaminyl-6-O-methyl erythronolide A (Compound No. 11)
3-O-(3-Fluorophenyl)acetyl-5-O-(3′-N-desmethyl-3′-N-benzyl)desosaminyl-6-O-methyl erythronolide A (Compound No. 12)
3-O-(4-Fluorophenyl)acetyl-5-O-(3′-N-desmethyl-3′-N-benzyl)desosaminyl-6-O-methyl erythronolide A (Compound No. 13)
3-O-(4-Fluorophenyl)acetyl-5-O-[3′-N-desmethyl-3′-N-(3,4-difluoro)benzyl]desosaminyl-6-O-methyl erythronolide (Compound No. 14)
3-O-(4-Fluorophenyl)acetyl-5-O-[3′-N-desmethyl-3′-N-cyclopropyl)desosaminyl-6-O-methyl erythronolide A (Compound No. 15)
3-O-(3-Fluorophenyl)acetyl-5-O-[3′-N-desmethyl-3′-N-cyclopropyl)desosaminyl-6-O-methyl erythronolide A (Compound No. 16)
3-O-(4-Fluorophenyl)acetyl-5-O-[3′-N-desmethyl-3′-N-(3-hydroxy)benzyl]desosaminyl-6-O-methyl erythronolide A (Compound No. 17)
3-O-(4-Fluorophenyl)acetyl-5-O-(3′-N-desmethyl-3′-N-propargyl)desosaminyl-6-O-methyl erythronolide A (Compound No. 18)
3-O-(3-Fluorophenyl)acetyl-5-O-(3′-N-desmethyl-3′-N-allyl)desosaminyl-6-O-methyl erythronolide A (Compound No. 19)
3-O-(4-Fluorophenyl)acetyl-5-O-(3′-N-desmethyl-3′-N-allyl)desosaminyl-6-O-methyl erythronolide A (Compound No. 20)
3-O-(2-Fluorophenyl)acetyl-5-O-(3′-N-desmethyl-3′-N-propargyl)desosaminyl-6-O-methyl erythronolide A (Compound No. 21)
3-O-(3-Fluorophenyl)acetyl-5-O-(3′-N-desmethyl-3′-N-propargyl)desosaminyl-6-O-methyl erythronolide A (Compound No. 22)
3-O-(2-Fluorophenyl)acetyl-5-O-(3′-N-desmethyl-3′-N-allyl)desosaminyl-6-O-methyl erythronolide A (Compound No. 23)
3-O-(4-Fluorophenyl)acetyl-5-O-(3′-N-desmethyl-3′-N-(4-nitro)benzyl)desosaminyl-6-O-methyl erythronolide A (Compound No. 24)
3-O-(2-Nitrophenyl)acetyl-5-O-(3′-N-desmethyl-3′-N-cyclopropylmethyl)desosaminyl-6-O-methyl erythronolide A (Compound No. 25)
3-O-(4-Nitrophenyl)acetyl-5-O-(3′-N-desmethyl-3′-N-cyclopropylmethyl)desosaminyl-6-O-methyl erythronolide A (Compound No. 26)
3-O-(3-Nitrophenyl)acetyl-5-O-(3′-N-desmethyl-3′-N-benzyl)desosaminyl-6-O-methyl erythronolide A (Compound No. 27)
3-O-(2-Nitrophenyl)acetyl-5-O-(3′-N-desmethyl-3′-N-ethyl)desoaminyl-6-O-methyl erythronolide A (Compound No. 28)
3-O-(3-Nitrophenyl)acetyl-5-O-(3′-N-desmethyl-3′-N-ethyl)desoaminyl-6-O-methyl erythronolide A (Compound No. 29)
3-O-(4-Nitrophenyl)acetyl-5-O-(3′-N-desmethyl-3′-N-ethyl)desoaminyl-6-O-methyl erythronolide A (Compound No. 30)
3-O-(2-Nitrophenyl)acetyl-5-O-(3′-N-desmethyl-3′-N-allyl)desosaminyl-6-O-methyl erythronolide A (Compound No. 31)
3-O-(4-Nitrophenyl)acetyl-5-O-(3′-N-desmethyl-3′-N-allyl)desosaminyl-6-O-methyl erythronolide A (Compound No. 32)
3-O-(3-Nitrophenyl)acetyl-5-O-(3′-N-desmethyl-3′-N-allyl)desosaminyl-6-O-methyl erythronolide A (Compound No. 33)
3-O-(2-Nitrophenyl)acetyl-5-O-(3′-N-desmethyl-3′-N-propargyl)desosaminyl-6-O-methyl erythronolide A (Compound No. 34)
3-O-(3-Nitrophenyl)acetyl-5-O-(3′-N-desmethyl-3′-N-propargyl)desosaminyl-6-O-methyl erythronolide A (Compound No. 35)
3-O-(2-Nitrophenyl)acetyl-5-O-(3′-N-desmethyl-3′-N-isopropyl)desosaminyl-6-O-methyl erythronolide A (Compound No. 36)
3-O-(3-Nitrophenyl)acetyl-5-O-(3′-N-desmethyl-3′-N-cyclopropyl)desosaminyl-6-O-methyl erythronolide A (Compound No. 37)
3-O-(4-Nitrophenyl)acetyl-5-O-(3′-N-desmethyl-3′-N-cyclopropyl)desosaminyl-6-O-methyl erythronolide A (Compound No. 38)
3-O-(2-Pyridyl)acetyl-5-O-(31-N-desmethyl-3′-N-cyclopropylmethyl)desosaminyl-6-O-methyl erythronolide A (Compound No. 39)
3-O-(4-Pyridyl)acetyl-5-O-(3′-N-desmethyl-3′-N-cyclopropylmethyl)desosaminyl-6-O-methyl erythronolide A (Compound No. 40)
3-O-(2-Pyridyl)acetyl-5-O-(3′-N-desmethyl-3′-N-ethyl desoaminyl-6-O-methyl erythronolide A (Compound No. 41)
3-O-(3-Pyridyl)acetyl-5-O-(3′-N-desmethyl-3′-N-benzyl)desosaminyl-6-O-methyl erythronolide A (Compound No. 42)
3-O-(2-Pyridyl)acetyl-5-O-[3′-N-desmethyl-3′-N-benzyl]desosaminyl-6-O-methyl erythronolide A (Compound No. 43)
3-O-(2-Pyridyl)acetyl-5-O-(3′-N-desmethyl-3′-N-allyl)desosaminyl-6-O-methyl erythronolide A (Compound No. 44)
3-O-(3-Pyridyl)acetyl-5-O-(3′-N-desmethyl-3′-N-allyl)desosaminyl-6-O-methyl erythronolide A (Compound No. 45)
3-O-(2-Pyridyl)acetyl-5-O-(3′-N-desmethyl-3′-N-propargyl)desosaminyl-6-O-methyl erythronolide A (Compound No. 46)
3-O-(3-Pyridyl)acetyl-5-O-(3′-N-desmethyl-3′-N-propargyl)desosaminyl-6-O-methyl erythronolide A (Compound No. 47)
3-O-(4-Pyridyl)acetyl-5-O-(3′-N-desmethyl-3′-N-propargyl)desosaminyl-6-O-methyl erythronolide A (Compound No. 48)
3-O-(4-Pyridyl)acetyl-5-O-(3′-N-desmethyl-3′-N-cyclopropyl)desosaminyl-6-O-methyl erythronolide A (Compound No. 49)
3-O-(2-Pyridyl)acetyl-5-O-(3′-N-desmethyl-3′-N-cyclopropylmethyl)desosaminyl-6-O-methyl erythronolide A (Compound No. 50)
3-O-(4-Pyridyl)acetyl-5-O-(3′-N-desmethyl-3′-N-ethyl)desosaminyl-6-O-methyl erythronolide A (Compound No. 51)
3-O-(3-Pyridyl)acetyl-5-O-(3′-N-desmethyl-3′-N-ethyl)desosaminyl-6-O-methyl erythronolide A (Compound No. 52)
3-O-(4-Pyridyl)acetyl-5-O-N-desmethyl-3′-N-cyclopropylmethyl)desosaminyl-6-O-methyl erythronolide A (Compound No. 53)
3-O-(4-Pyridyl)acetyl-5-O-(3′-N-desmethyl-3′-N-allyl)desosaminyl-6-O-methyl erythronolide A (Compound No. 54)
3-O-(2-Pyridyl)acetyl-5-O-(3′-N-desmethyl-3′-N-cyclopropyl)desosaminyl-6-O-methyl erythronolide A (Compound No. 55)
3-O-(Phenyl)acetyl-5-O-[(3′-N-desmethyl-3′-N-cyclopropylmethyl]desoaminyl-6-O-methyl erythronolide A (Compound No. 56)
3-O-(Phenyl)acetyl-5-O-(3′-N-desmethyl-3′-N-(4-fluoro)benzyl)desoaminyl-6-O-methyl erythronolide A (Compound No. 57)
3-O-(Phenyl)acetyl-5-O-(3′-N-desmethyl-3′-N-benzyl)desoaminyl-6-O-methyl erythronolide A (Compound No. 58)
3-O-(2-Thiophene)acetyl-5-O-(3′-N-desmethyl-3′-N-cyclopropylmethyl)desosaminyl-6-O-methyl erythronolide A (Compound No. 59)
3-O-(2-Thiophene)acetyl-5-O-(3′-N-desmethyl-3′-N-ethyl)desoaminyl-6-O-methyl erythronolide A (Compound No. 60)
3-O-(2-Thiophene)acetyl-5-O-(3′-N-desmethyl-3′-N-benzyl)desosaminyl-6-O-methyl erythronolide A (Compound No. 61)
3-O-(2-Thiophene)acetyl-5-O-[3′-N-desmethyl-3′-N-(3-hydroxy)benzyl]desosaminyl-6-O-methyl erythronolide A (Compound No. 62)
3-O-(4-Chlorophenyl)acetyl-5-O-(3′-N-desmethyl-3′-N-ethyl)desoaminyl-6-O-methyl erythronolide A (Compound No. 63)
3-O-(4-Chlorophenyl)acetyl-5-O-(3′-N-desmethyl-3′-N-benzyl)desosaminyl-6-O-methyl erythronolide A (Compound No. 64)
3-O-(4-Chlorophenyl)acetyl-5-O-[3′-N-desmethyl-3′-N-(3-hydroxy)benzyl]desosaminyl-6-O-methyl erythronolide A (Compound No. 65)
3-O-(2-Methylphenyl)acetyl-5-O-[3′-N-desmethyl-3′-N-(4-hydroxy)benzyl]desosaminyl-6-O-methyl erythronolide A (Compound No. 66)
3-O-(2-Methylphenyl)acetyl-5-O-(3′-N-desmethyl-3′-N-benzyl)desosaminyl-6-O-methyl erythronolide A (Compound No. 67)
3-O-(4-Methylphenyl)acetyl-5-O-(3′-N-desmethyl-3′-N-benzyl)desosaminyl-6-O-methyl erythronolide A (Compound No. 68)
3-O-(4-Methoxyphenyl)acetyl-5-O-(3′-N-desmethyl-3′-N-cyclopropylmethyl)desosaminyl-6-O-methyl erythronolide A (Compound No. 69)
3-O-(4-Methoxyphenyl)acetyl-5-O-[3′-N-desmethyl-3′-N-(3,4-difluoro)benzyl]desosaminyl-6-O-methyl erythronolide A (Compound No. 70)
3-O-(1-Naphthyl)acetyl-5-O-[3′-N-desmethyl-3′-N-(4-hydroxy)benzyl]desosaminyl-6-O-methyl erythronolide A (Compound No. 71)
3-O-(1-Naphthyl)acetyl-5-O-(3′-N-desmethyl-3′-N-benzyl)desosaminyl-6-O-methyl erythronolide A (Compound No. 72)
3-O-(2-Naphthyl)acetyl-5-O-(3′-N-desmethyl-3′-N-benzyl)desosaminyl-6-O-methyl erythronolide A (Compound No. 73)
3-O-(2,4-Difluorophenyl)acetyl-5-O-(3′-N-desmethyl-3′-N-benzyl)desosaminyl-6-O-methyl erythronolide A (Compound No. 74)
3-O-(2,4-Difluorophenyl)acetyl-5-O-[3′-N-desmethyl-3′-N-(3,4-difluoro)benzyl]desosaminyl-6-O-methyl erythronolide A (Compound No. 75)
3-O-(2-Bromophenyl)acetyl-5-O-[3′-N-desmethyl-3′-N-(4-hydroxy)benzyl]desosaminyl-6-O-methyl erythronolide A (Compound No. 76)
3-O-(2-Bromophenyl)acetyl-5-O-(3′-N-desmethyl-3′-N-benzyl)desosaminyl-6-O-methyl erythronolide A (Compound No. 77)
3-O-(3-Indole)acetyl-5-O-(3′-N-desmethyl-3′-N-ethyl)desoaminyl-6-O-methyl erythronolide A (Compound No. 78)
3-O-(2-Napthyl)acetyl-5-O-(3′-N-desmethyl-3′-N-ethyl)desoaminyl-6-O-methyl erythronolide A (Compound No. 79)

3. A pharmaceutical composition comprising a pharmaceutically effective amount of a compound as defined in claim 1 and 2 together with pharmaceutically acceptable carriers, excipients, or diluents.

4. A method for treating or preventing an animal or human suffering from bacterial infection caused by gram positive or gram negative or atypical pathogens comprising administering to a mammal in need of such treatment a pharmaceutically effective amount of a compound having the structure of Formula I, 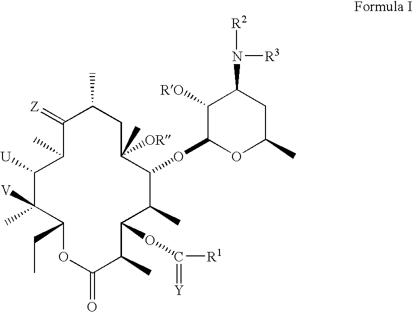
and its pharmaceutically acceptable acid addition salts, pharmaceutically acceptable solvates, enantiomers, diastereomers, polymorphs and metabolites, wherein
R¹ represents: lower alkyl (C₁-C₅) group, lower alkyl (C₁-C₅) having one or more halogen (F, Cl, Br, I) atoms as substituent (s), lower alkyl (C₁-C₅) amino group, lower alkyl amino (C₁-C₅) carbonyl group; lower alkoxy group (C₁-C₅); or five or six membered aryl or heteroaryl ring having 1 to 3 hetero atoms selected from the group consisting of oxygen, nitrogen, and sulphur, wherein the aryl or heteroaryl ring may be unsubstituted or substituted by 1 to 3 substituents independently selected from the group consisting of lower alkyl (C₁-C₅) group, lower alkyl (C₁-C₅) group having one or more halogen (F, Cl, Br, I) atoms, lower alkoxy (C₁-C₅) groups, lower alkyl (C₁-C₅) amino group, halogen atoms (F, Cl, Br, I), amino group, nitro group, hydroxy group, and cyano group;
R² and R³ are independently selected from: C₁-C₆ alkyl group optionally substituted with halogen atoms (F, Cl, Br, I); cycloalkyl (C₃-C₇) group; or five to six membered aryl or heteroaryl ring having 1 to 3 hetero atom independently selected from the group consisting of nitrogen, oxygen, and sulphur, wherein the aryl or heteroaryl ring may be unsubstituted or substituted by 1 to 3 substituents independently selected from the group consisting of lower alkyl (C₁-C₃), lower alkyl (C₁-C₃) group having one or more halogen (F, Cl, Br, I) atom as substituent(s), lower alkoxy (C₁-C₃) group, lower alkyl (C₁-C₃) amino group, halogen (F, Cl, Br, I) atoms, nitro group, hydroxy group, or cyano group; the above-mentioned C₁-C₆ alkyl group may be substituted by: NHCOR⁵, NHCOOR⁵, OCOR⁵, COR⁵ wherein R⁵ represents lower alkyl (C₁-C₅); five to six membered aryl or heteroaryl ring having 1 to 3 hetero atom independently selected from the group consisting of nitrogen, oxygen, and sulphur, wherein the aryl or heteroaryl ring may be unsubstituted or substituted by 1 to 3 substituents independently selected from the group consisting of lower alkyl (C₁-C₃), lower alkyl (C₁-C₃) group having one or more halogen (F, Cl, Br, I) atoms as substituent(s), lower alkoxy (C₁-C₃) group, lower alkyl (C₁-C₃) amino group, halogen (F, Cl, Br, I) atoms, nitro group, hydroxy group, and cyano group; C₂-C₆ alkenyl or alkyne group optionally substituted with halogen (F, Cl, Br, I) atoms or a group consisting of NHCOR⁵, NHCOOR⁵, COR⁵, OCOR⁵ (wherein R⁵ is as defined above); cycloalkyl (C₃-C₇) group; five or six membered aryl or heteroaryl ring having 1 to 3 hetero atom independently selected from the group consisting of nitrogen, oxygen, and sulphur, wherein the aryl or heteroaryl ring may be unsubstituted or substituted by 1 to 3 substituents independently selected from the group consisting of lower alkyl (C₁-C₃) group, lower alkyl (C₁-C₃) group having one or more halogen (F, Cl, Br, I) atoms as substituent(s), lower alkoxy (C₁-C₃) group, lower alkyl (C₁-C₃) amino, halogen (F, Cl, Br, I) atoms, nitro group, hydroxy group, amino group, and cyano group;
R′ represents hydrogen, or a hydroxy protecting group optionally selected from acetyl, benzoyl, butyldiphenylsilyl, methylthiomethyl, or methoxy methyl;
R″ represents hydrogen, or a lower alkyl (C₁-C₃) group;
Y represents oxygen or sulphur;
Z represents an oxygen atom or a group represented by NOR⁶, wherein R⁶ represents hydrogen atom, alkyl (C₁-C₆) group, alkyl (C₁-C₆) amino group, phenyl or benzyl group, or phenyl or benzyl group having 1 to 5 substituent independently selected from halogen (F, Cl, Br, I) atoms, lower alkyl (C₁-C₃) group, hydroxy group, nitro group, cyano group, or amino group;
U represents a hydroxy group: OR⁷, wherein R⁷ represents hydroxy protecting group selected from acetyl, benzoyl, butyldiphenylsilyl, methylthiomethyl, or methoxymethyl; or —NH(CH₂)ₙR⁸, wherein n represents 0 to 4 and R⁸ represents five or six membered aryl or heteroaryl ring having 1 to 4 hetero atom independently selected from the group consisting of nitrogen, oxygen, and sulphur, wherein the aryl or heteroaryl ring may be unsubstituted or substituted by one to three substituents independently selected from the group consisting of lower alkyl (C₁-C₃) group, lower alkyl (C₁-C₃) group having one or more halogen (F, Cl, Br, I) atoms as substituent (s), lower alkoxy (C₁-C₃) group, lower alkyl (C₁-C₃) amino, halogen (F, Cl, Br, I) atoms, nitro group, hydroxy group, amino group, and cyano group;
V represents: hydrogen atom; hydroxy group; or OR⁷, wherein R⁷ represents a hydroxy protecting group selected from the group consisting of acetyl, benzoyl, butyldiphenylsilyl, methylthiomethylm and methoxymethyl;
U and V may also together represent (with carbon atoms at the 11- and 12-positions on the erythronolide skeleton): a group represented by Formula 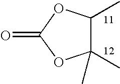
or a group represented by the Formula 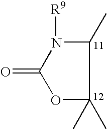
wherein R⁹ represents: hydrogen atom; alkyl (C₁-C₆) group, wherein the alkyl (C₁-C₆) may be unsubstituted or substituted by halogen (F, Cl, Br, I) atoms, five or six membered aryl or heteroaryl ring having 1 to 3 hetero atom independently selected from the group consisting of nitrogen, oxygen, and sulphur, wherein the aryl or heteroaryl ring may be unsubstituted or substituted by 1 to 3 substituents independently selected from the group consisting of lower alkyl (C₁-C₃) group, lower alkyl (C₁-C₃) group having one or more halogen (F, Cl, Br, I) atoms as substituent(s), lower alkoxy (C₁-C₃) group, lower alkyl (C₁-C₃) amino, halogen (F, Cl, Br, I) atoms, nitro group, hydroxy group, amino group, and cyano group.

5. A method for treating or preventing of animal or human suffering from bacterial infections according to claim 4 caused by bacteria selected from the group consisting of Staphylococcus aureus, Streptococcus pneumoniae, Enterococcus faecalis, Escherichia coli, Pseudomonas aeruginosa, and Haemophilus influenzae.

6. A method for treating or preventing an animal or human suffering from bacterial infection caused by gram positive or gram negative or atypical pathogens comprising administering to a mammal in need of such treatment therapeutically effective amount of a pharmaceutical composition according to claim 3.

7. A method for treating or preventing of animal or human suffering from bacterial infections caused by bacteria selected from the group consisting of Staphylococcus aureus, Streptococcus pneumoniae, Enterococcus faecalis, Escherichia coli, Pseudomonas aeruginosa, and Haemophilus influenzae, comprising administering to a mammal in need of such treatment therapeutically amount of a pharmaceutical composition according to claim 3.

8. A process for preparing a compound of Formula I 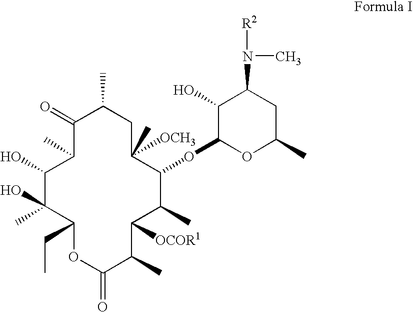
and its pharmaceutically acceptable acid addition salts, pharmaceutically acceptable solvates, enantiomers, diastereomers, polymorphs and metabolites, wherein
R³═R″═CH₃, R′═H, U═V═OH, and Y=Z=O
R¹ represents: lower alkyl (C₁-C₅) group, lower alkyl (C₁-C₅) having one or more halogen (F, Cl, Br, I) atoms as substituent (s), lower alkyl (C₁-C₅) amino group, lower alkyl amino (C₁-C₅) carbonyl group; lower alkoxy group (C₁-C₅); or five or six membered aryl or heteroaryl ring having 1 to 3 hetero atoms selected from the group consisting of oxygen, nitrogen, and sulphur, wherein the aryl or heteroaryl ring may be unsubstituted or substituted by 1 to 3 substituents independently selected from the group consisting of lower alkyl (C₁-C₅) group, lower alkyl (C₁-C₅) group having one or more halogen (F, Cl, Br, I) atoms, lower alkoxy (C₁-C₅) groups, lower alkyl (C₁-C₅) amino group, halogen atoms (F, Cl, Br, I), amino group, nitro group, hydroxy group, and cyano group;
R² and R³ are independently selected from: C₁-C₆ alkyl group optionally substituted with halogen atoms (F, Cl, Br, I); cycloalkyl (C₃-C₇) group; or five to six membered aryl or heteroaryl ring having 1 to 3 hetero atom independently selected from the group consisting of nitrogen, oxygen, and sulphur, wherein the aryl or heteroaryl ring may be unsubstituted or substituted by 1 to 3 substituents independently selected from the group consisting of lower alkyl (C₁-C₃), lower alkyl (C₁-C₃) group having one or more halogen (F, Cl, Br, I) atom as substituent(s), lower alkoxy (C₁-C₃) group, lower alkyl (C₁-C₃) amino group, halogen (F, Cl, Br, I) atoms, nitro group, hydroxy group, or cyano group; the above-mentioned C₁-C₆ alkyl group may be substituted by: NHCOR⁵, NHCOOR⁵, OCOR⁵, COR⁵ wherein R⁵ represents lower alkyl (C₁-C₅); five to six membered aryl or heteroaryl ring having 1 to 3 hetero atom independently selected from the group consisting of nitrogen, oxygen, and sulphur, wherein the aryl or heteroaryl ring may be unsubstituted or substituted by 1 to 3 substituents independently selected from the group consisting of lower alkyl (C₁-C₃), lower alkyl (C₁-C₃) group having one or more halogen (F, Cl, Br, I) atoms as substituent(s), lower alkoxy (C₁-C₃) group, lower alkyl (C₁-C₃) amino group, halogen (F, Cl, Br, I) atoms, nitro group, hydroxy group, and cyano group; C₂-C₆ alkenyl or alkyne group optionally substituted with halogen (F, Cl, Br, I) atoms or a group consisting of NHCOR⁵, NHCOOR⁵, COR⁵, OCOR⁵ (wherein R⁵ is as defined above); cycloalkyl (C₃-C₇) group; five or six membered aryl or heteroaryl ring having 1 to 3 hetero atom independently selected from the group consisting of nitrogen, oxygen, and sulphur, wherein the aryl or heteroaryl ring may be unsubstituted or substituted by 1 to 3 substituents independently selected from the group consisting of lower alkyl (C₁-C₃) group, lower alkyl (C₁-C₃) group having one or more halogen (F, Cl, Br, I) atoms as substituent(s), lower alkoxy (C₁-C₃) group, lower alkyl (C₁-C₃) amino, halogen (F, Cl, Br, I) atoms, nitro group, hydroxy group, amino group, and cyano group;
R′ represents hydrogen, or a hydroxy protecting group optionally selected from acetyl, benzoyl, butyldiphenylsilyl, methylthiomethyl, or methoxy methyl;
R″ represents hydrogen, or a lower alkyl (C₁-C₃) group;
Y represents oxygen or sulphur;
Z represents an oxygen atom or a group represented by NOR⁶, wherein R⁶ represents hydrogen atom, alkyl (C₁-C₆) group, alkyl (C₁-C₆) amino group, phenyl or benzyl group, or phenyl or benzyl group having 1 to 5 substituent independently selected from halogen (F, Cl, Br, I) atoms, lower alkyl (C₁-C₃) group, hydroxy group, nitro group, cyano group, or amino group;
U represents a hydroxy group: OR⁷, wherein R⁷ represents hydroxy protecting group selected from acetyl, benzoyl, butyldiphenylsilyl, methylthiomethyl, or methoxymethyl; or —NH(CH₂)ₙR⁸, wherein n represents 0 to 4 and R⁸ represents five or six membered aryl or heteroaryl ring having 1 to 4 hetero atom independently selected from the group consisting of nitrogen, oxygen, and sulphur, wherein the aryl or heteroaryl ring may be unsubstituted or substituted by one to three substituents independently selected from the group consisting of lower alkyl (C₁-C₃) group, lower alkyl (C₁-C₃) group having one or more halogen (F, Cl, Br, I) atoms as substituent (s), lower alkoxy (C₁-C₃) group, lower alkyl (C₁-C₃) amino, halogen (F, Cl, Br, I) atoms, nitro group, hydroxy group, amino group, and cyano group;
V represents: hydrogen atom; hydroxy group; or OR⁷, wherein R⁷ represents a hydroxy protecting group selected from the group consisting of acetyl, benzoyl, butyldiphenylsilyl, methylthiomethylm and methoxymethyl;
U and V may also together represent (with carbon atoms at the 11- and 12-positions on the erythronolide skeleton): a group represented by Formula 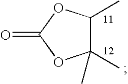
or a group represented by the Formula 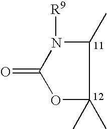
wherein R⁹ represents: hydrogen atom; alkyl (C₁-C₆) group, wherein the alkyl (C₁-C₆) may be unsubstituted or substituted by halogen (F, Cl, Br, I) atoms, five or six membered aryl or heteroaryl ring having 1 to 3 hetero atom independently selected from the group consisting of nitrogen, oxygen, and sulphur, wherein the aryl or heteroaryl ring may be unsubstituted or substituted by 1 to 3 substituents independently selected from the group consisting of lower alkyl (C₁-C₃) group, lower alkyl (C₁-C₃) group having one or more halogen (F, Cl, Br, I) atoms as substituent(s), lower alkoxy (C₁-C₃) group, lower alkyl (C₁-C₃) amino, halogen (F, Cl, Br, I) atoms, nitro group, hydroxy group, amino group, and cyano group, which method comprises:
Step (1) treating clarithromycin of Formula II 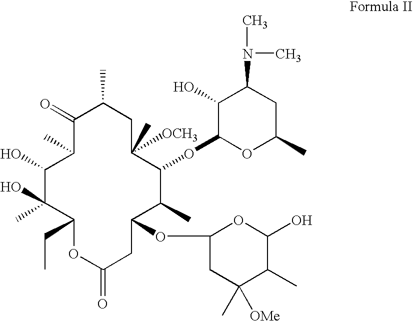
with an acid at ambient temperature to give a compound of Formula III, 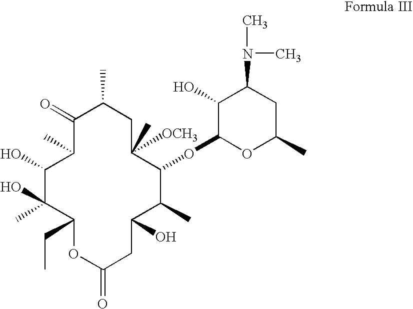
Step (2) reacting the compound of Formula I with a reagent of Formula R′₂O or R′X (wherein R′ is hydroxy protecting group optionally selected from acetyl, benzoyl, butyldiphenylsilyl, methylthiomethyl, methoxy methyl and X is an optional halogen atom) to give a compound of Formula IV, 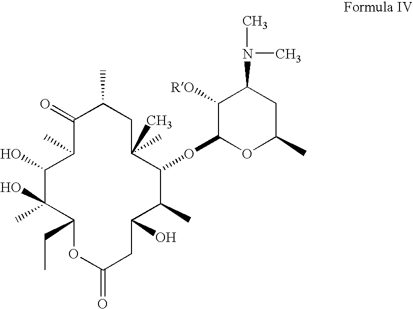
Step (3) reacting the compound of Formula IV with a reagent of Formula R¹COOH, R¹COX, (R¹CO)₂O or R¹COOR⁴ (wherein R¹ is as defined for Formula I in claim 1 and R⁴ is a group selected from pivaloyl group, p-toluenesulfonyl group, isobutoxycarbonyl group, ethoxycarbonyl group or isopropoxycarbonyl group) to give a compound of Formula V, 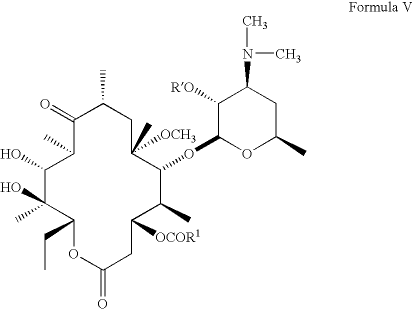
Step (4) treating the compound of Formula V with aqueous alcohol to give a compound of Formula VI, 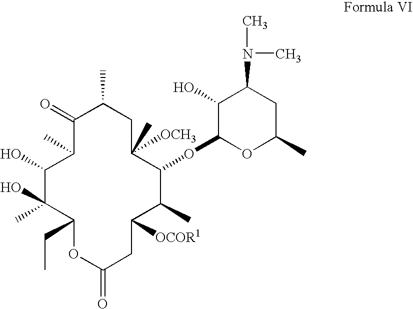
Step (5) desmethylating at 3′-N-dimethyl group of the compound of Formula VI with N-iodosuccinamide and acetonitrile or iodine in presence of sodium acetate followed by quench with sodium thiosulphate to give a compound of Formula VII, 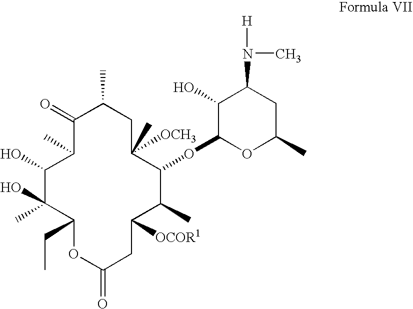
Step (6) reacting the compound of Formula VII with a reagent of Formula R²CHO or R²₂CO (wherein R² is as defined for Formula 1 in claim 1) to give a compound of Formula I 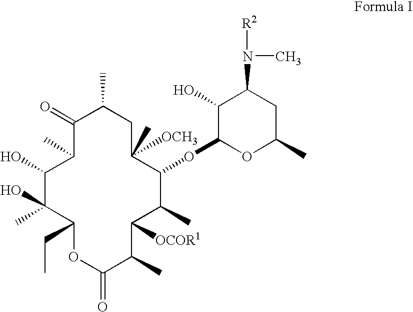
R³═R″═CH₃, R′═H, U═V═OH, and Y=Z=O

9. The process according to claim 8 wherein, the reaction of clarithromycin of Formula II with hydrochloric or dichloroacetic acid to give a compound of Formula III is carried out in presence of aqueous alcohol selected from the group comprising of aqueous methanol, aqueous ethanol, aqueous propanol and aqueous isopropanol.

10. The process according to claim 8 wherein, the reaction of compound of Formula III with a reagent of Formula R′₂O or R′X to give a compound of Formula IV is carried out in presence of an inorganic base selected from the group comprising of sodium hydrogen carbonate, potassium carbonate or an organic base selected from the group comprising of triethylamine, pyridine, tributylamine and 4-N-dimethylaminopyridine.

11. The process according to claim 8 wherein, the reaction of compound of Formula III with a reagent of Formula R′₂O or R′X to give a compound of Formula IV is carried out in presence of an inert solvent selected from the group comprising of dichloromethane, dichloroethane, acetone, ethyl acetate and tetrahydrofuran.

12. The process according to claim 8 wherein, the reaction of compound of Formula IV with a reagent of Formula R¹COOH, R¹COX, (R¹CO)₂O or R¹COOR⁴ to give a compound of Formula V is carried out in presence of an activating agent selected from the group comprising of dichlorohexylcarbodiimide (DCC) and 1-ethyl-3(3-dimethylaminopropyl)carbodiimide hydrochloride (EDCI).

13. The process according to claim 8 wherein, the reaction of compound of Formula IV with a reagent of Formula R¹COOH, R¹COX, (R¹CO)₂O or R¹COOR⁴ to give a compound of Formula V is carried out in presence of an inorganic base selected from the group comprising of sodium hydrogen carbonate, potassium carbonate or organic base selected from the group comprising of triethylamine, pyridine, tributylamine and 4-dimethylaminopyridine.

14. The process according to claim 8 wherein, the reaction of compound of Formula IV with a reagent of Formula R¹COOH, R¹COX, (R¹CO)₂O or R¹COOR⁴ to give a compound of Formula V is carried out in presence of an inert solvent selected from the group comprising of dichloromethane, dichloroethane, acetone, ethyl acetate and tetrahydrofuran.

15. The process according to claim 8 wherein, the reaction of compound of Formula V is carried out with aqueous alcohol selected from the group comprising of aqueous methanol, aqueous ethanol, aqueous propanol and aqueous isopropanol to give a compound of Formula VI.

16. The process according to claim 8 wherein, the reaction of the compound of Formula VII with a reagent of Formula R²CHO or R²₂CO to give a compound of Formula I is carried out in presence of a reducing agent selected from the group comprising of sodium borohydride, sodium cyanoborohydride, sodium triacetoborohydride or palladium/carbon catalyst.

17. The process according to claim 8 wherein, the reaction of the compound of Formula VII with a reagent of Formula R²CHO or R²₂CO to give a compound of Formula I is carried out in presence of a protic or non-protic solvent selected from the group comprising of hexane, toluene, methylene chloride, ethylene chloride, chloroform, tetrahydrofuran, N-methyl-pyrrolidinone, diethyl ether, bis-methoxymethyl ether, dimethylformamide, acetonitrile, acetone and ethyl acetate.

18. A Process for preparing a compound of Formula I 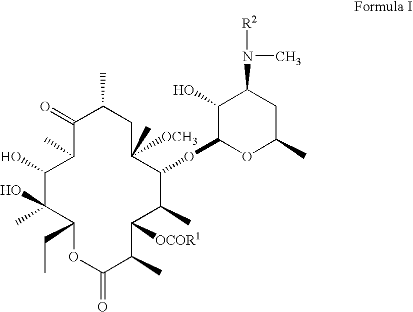
and its pharmaceutically acceptable acid addition salts, pharmaceutically acceptable solvates, enantiomers, diastereomers, polymorphs and metabolites, wherein
R³═R″═CH₃, R′═H, U═V═OH, Y=Z=O
R¹ represents lower alkyl (C₁-C₅) group, lower alkyl (C₁-C₅) having one or more halogen (F, Cl, Br, I) atoms as substituent (s), lower alkyl (C₁-C₅) amino group, lower alkyl amino (C₁-C₅) carbonyl group, lower alkoxy group (C₁-C₅), five or six membered aryl or heteroaryl ring having 1 to 3 hetero atoms selected from the group consisting of oxygen, nitrogen, sulphur, the aryl or heteroaryl ring may be unsubstituted or substituted by 1 to 3 substituent independently selected from the group consisting of lower alkyl (C₁-C₅) group, lower alkyl (C₁-C₅) group having one or more halogen (F, Cl, Br, I) atoms, lower alkoxy (C₁-C₅) groups, lower alkyl (C₁-C₅) amino group, halogen atoms (F, Cl, Br, I), amino group, nitro group, hydroxy group, cyano group;
R² is selected from C₁-C₆ alkyl group optionally substituted with halogen atoms (F, Cl, Br, I), cycloalkyl (C₃-C₇) group, five to six membered aryl or heteroaryl ring having 1 to 3 hetero atom independently selected from the group consisting of nitrogen, oxygen, sulphur, the aryl or heteroaryl ring may be unsubstituted or substituted by 1 to 3 substituent independently selected from the group consisting of lower alkyl (C₁-C₃), lower alkyl (C₁-C₃) group having one or more halogen (F, Cl, Br, I) atom as substituent (s), lower alkoxy (C₁-C₃) group, lower alkyl (C₁-C₃) amino group, halogen (F, Cl, Br, I) atoms, nitro group, hydroxy group, cyano group, C₁-C₆ alkyl group may also be substituted by a group consisting of NHCOR⁵, NHCOOR⁵, OCOR⁵, COR⁵ [wherein R⁵ represents lower alkyl (C₁-C₅), five to six membered aryl or heteroaryl
ring having 1 to 3 hetero atom independently selected from the group consisting of nitrogen, oxygen, sulphur, the aryl or heteroaryl ring may be unsubstituted or substituted by 1 to 3 substituent independently selected from the group consisting of lower alkyl (C₁-C₃), lower alkyl (C₁-C₃) group having one or more halogen (F, Cl, Br, I) atoms as substituent (s), lower alkoxy (C₁-C₃) group, lower alkyl (C₁-C₃) amino group, halogen (F, Cl, Br, I) atoms, nitro group, hydroxy group, cyano group]; C₂-C₆ alkenyl or alkyne group optionally substituted with halogen (F, Cl, Br, I) atoms or a group consisting of NHCOR⁵, NHCOOR⁵, COR⁵, OCOR⁵ (wherein R⁵ is as defined above); cycloalkyl (C₃-C₇) group; five or six membered aryl or heteroaryl ring having 1 to 3 hetero atom independently selected from the group consisting of nitrogen, oxygen, sulphur, the aryl or heteroaryl ring may be unsubstituted or substituted by 1 to 3 substituents independently selected from the group consisting of lower alkyl (C₁-C₃) group, lower alkyl (C₁-C₃) group having one or more halogen (F, Cl, Br, I) atoms as substituent (s), lower alkoxy (C₁-C₃) group, lower alkyl (C₁-C₃) amino, halogen (F, Cl, Br, I) atoms, nitro group, hydroxy group, amino group, cyano group, which method comprises the steps of
Step (1) desmethylating at 3′-N-dimethyl group of the compound of Formula II with N-iodosuccinamide and acetonitrile or iodine in presence of sodium acetate followed by quench with sodium thiosulphate to give a compound of Formula VIII 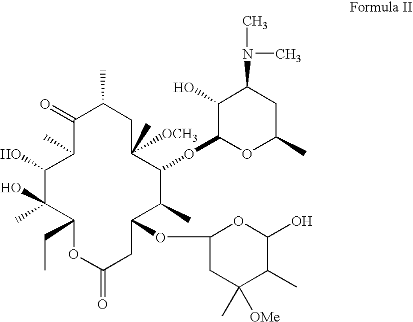 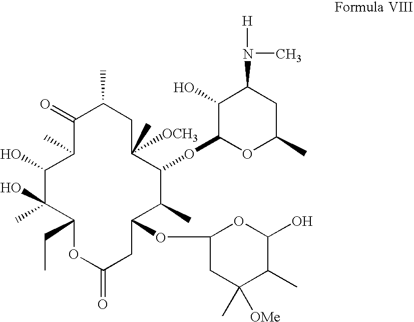
Step (2) reacting the compound of Formula VIII with a reagent of Formula R²CHO or R²₂CO (wherein R² is as defined for Formula 1 in claim 1) to give a compound of Formula IX 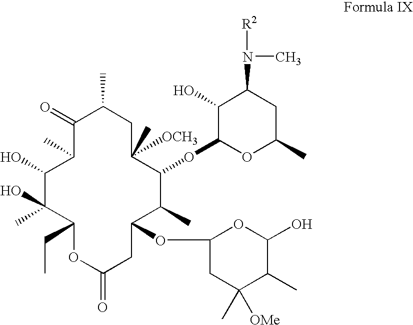
Step (3) treating the compound of Formula IX with acid at an ambient temperature to give a compound of Formula X 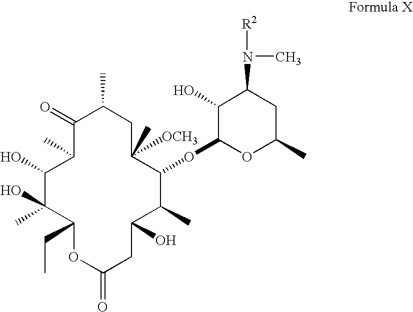
Step (4) reacting the compound of Formula X with a reagent of Formula R′₂O or R′X (wherein R′ is hydroxy protecting group optionally selected from acetyl, benzoyl, butyldiphenylsilyl, methylthiomethyl, methoxy methyl and X is an optional halogen atom) to give a compound of Formula XI 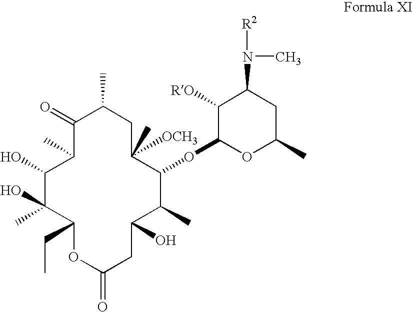
Step (5) reacting the compound of Formula XI with a reagent of Formula R¹COOH, R¹COX, (R¹CO)₂O or R¹COOR⁴ (wherein R¹ is as defined for Formula I in claim 1 and R⁴ is a group selected from pivaloyl group, p-toluenesulfonyl group, isobutoxycarbonyl group, ethoxycarbonyl group or isopropoxycarbonyl group) to give a compound of Formula XII 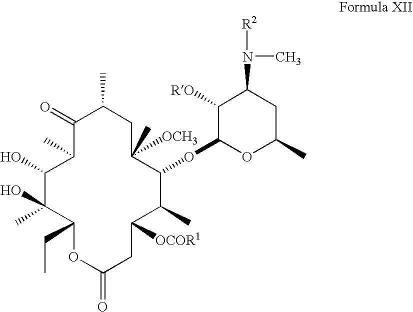
Step (6) treating the compound of Formula XII with aqueous alcohol to give a compound of Formula I 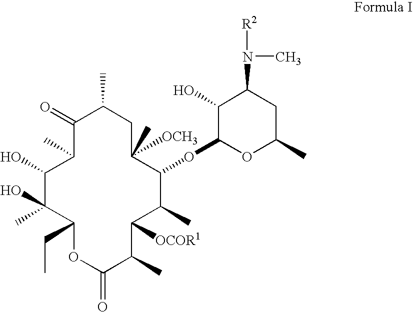
R³═R″═CH₃, R′═H, U═V═OH, and Y=Z=O

19. The process according to claim 18 wherein, the reaction of the compound of Formula VIII with a reagent of Formula R²CHO or R²₂CO to give a compound of Formula IX is carried out in presence of a reducing agent selected from the group comprising of sodium borohydride, sodium cyanoborohydride, sodium triacetoborohydride or palladium/carbon catalyst.

20. The process according to claim 18 wherein, the reaction of the compound of Formula VIII with a reagent of Formula R²CHO or R²₂CO to give a compound of Formula IX is carried out in presence of a protic or non-protic solvent selected from the group comprising of hexane, toluene, methylene chloride, ethylene chloride, chloroform, tetrahydrofuran, N-methyl-pyrrolidinone, diethyl ether, bis-methoxymethyl ether, dimethylformamide, acetonitrile, acetone and ethyl acetate.

21. The process according to claim 18 wherein, the reaction of compound of Formula IX with hydrochloric or dichloroacetic acid is carried out with aqueous alcohol selected from the group comprising of aqueous methanol, aqueous ethanol, aqueous propanol and aqueous isopropanol to give a compound of Formula X.

22. The process according to claim 18 wherein, the reaction of compound of Formula X with a reagent of Formula R′₂O or R′X to give a compound of Formula XI is carried out in presence of an inorganic base selected from the group comprising of sodium hydrogen carbonate, potassium carbonate or an organic base selected from the group comprising of triethylamine, pyridine, tributylamine and 4-N-dimethylaminopyridine.

23. The process according to claim 18 wherein, the reaction of compound of Formula X with a reagent of Formula R′₂O or R′X to give a compound of Formula XI is carried out in presence of an inert solvent selected from the group comprising of dichloromethane, dichloroethane, acetone, ethyl acetate and tetrahydrofuran.

24. The process according to claim 18 wherein, the reaction of compound of Formula XI with a reagent of Formula R¹COOH, R¹COX, (R¹CO)₂O or R¹COOR⁴ to give a compound of Formula XII is carried out in presence of an activating agent selected from the group comprising of dichlorohexylcarbodiimide (DCC) and 1-ethyl-3(3-dimethylaminopropyl)carbodiimide hydrochloride (EDCI).

25. The process according to claim 18 wherein, the reaction of compound of Formula XI with a reagent of Formula R¹COOH, R¹COX, (R¹CO)₂O or R¹COOR⁴ to give a compound of Formula XII is carried out in presence of an inorganic base selected from the group comprising of sodium hydrogen carbonate, potassium carbonate or organic base selected from the group comprising of triethylamine, pyridine, tributylamine and 4-dimethylaminopyridine.

26. The process according to claim 18 wherein, the reaction of compound of Formula XI with a reagent of Formula R¹COOH, R¹COX, (R¹CO)₂O or R¹COOR⁴ to give a compound of Formula XII is carried out in presence of an inert solvent selected from the group comprising of dichloromethane, dichloroethane, acetone, ethyl acetate and tetrahydrofuran.

27. The process according to claim 18 wherein, the reaction of compound of Formula XII is carried out with aqueous alcohol selected from the group comprising of aqueous methanol, aqueous ethanol, aqueous propanol and aqueous isopropanol to give a compound of Formula I.

28. A process for preparing a compound of Formula I 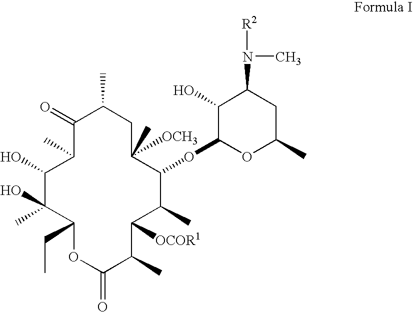
and its pharmaceutically acceptable acid addition salts, pharmaceutically acceptable solvates, enantiomers, diastereomers, polymorphs and metabolites, wherein
R³═R″═CH₃, R′═H, U═V═OH, Y=Z=O
R¹ represents lower alkyl (C₁-C₅) group, lower alkyl (C₁-C₅) having one or more halogen (F, Cl, Br, I) atoms as substituent (s), lower alkyl (C₁-C₅) amino group, lower alkyl amino (C₁-C₅) carbonyl group, lower alkoxy group (C₁-C₅), five or six membered aryl or heteroaryl ring having 1 to 3 hetero atoms selected from the group consisting of oxygen, nitrogen, sulphur, the aryl or heteroaryl ring may be unsubstituted or substituted by 1 to 3 substituent independently selected from the group consisting of lower alkyl (C₁-C₅) group, lower alkyl (C₁-C₅) group having one or more halogen (F, Cl, Br, I) atoms, lower alkoxy (C₁-C₅) groups, lower alkyl (C₁-C₅) amino group, halogen atoms (F, Cl, Br, I), amino group, nitro group, hydroxy group, cyano group;
R² is selected from C₁-C₆ alkyl group optionally substituted with halogen atoms (F, Cl, Br, I), cycloalkyl (C₃-C₇) group, five to six membered aryl or heteroaryl ring having 1 to 3 hetero atom independently selected from the group consisting of nitrogen, oxygen, sulphur, the aryl or heteroaryl ring may be unsubstituted or substituted by 1 to 3 substituent independently selected from the group consisting of lower alkyl (C₁-C₃), lower alkyl (C₁-C₃) group having one or more halogen (F, Cl, Br, I) atom as substituent (s), lower alkoxy (C₁-C₃) group, lower alkyl (C₁-C₃) amino group, halogen (F, Cl, Br, I) atoms, nitro group, hydroxy group, cyano group, C₁-C₆ alkyl group may also be substituted by a group consisting of NHCOR⁵, NHCOOR⁵, OCOR⁵, COR⁵ [wherein R⁵ represents lower alkyl (C₁-C₅), five to six membered aryl or heteroaryl ring having 1 to 3 hetero atom independently selected from the group consisting of nitrogen, oxygen, sulphur, the aryl or heteroaryl ring may be unsubstituted or substituted by 1 to 3 substituent independently selected from the group consisting of lower alkyl (C₁-C₃), lower alkyl (C₁-C₃) group having one or more halogen (F, Cl, Br, I) atoms as substituent (s), lower alkoxy (C₁-C₃) group, lower alkyl (C₁-C₃) amino group, halogen (F, Cl, Br, I) atoms, nitro group, hydroxy group, cyano group]; C₂-C₆ alkenyl or alkyne group optionally substituted with halogen (F, Cl, Br, I) atoms or a group consisting of NHCOR⁵, NHCOOR⁵, COR⁵, OCOR⁵ (wherein R⁵ is as defined above); cycloalkyl (C₃-C₇) group; five or six membered aryl or heteroaryl ring having 1 to 3 hetero atom independently selected from the group consisting of nitrogen, oxygen, sulphur, the aryl or heteroaryl ring may be unsubstituted or substituted by 1 to 3 substituents independently selected from the group consisting of lower alkyl (C₁-C₃) group, lower alkyl (C₁-C₃) group having one or more halogen (F, Cl, Br, I) atoms as substituent (s), lower alkoxy (C₁-C₃) group, lower alkyl (C₁-C₃) amino, halogen (F, Cl, Br, I) atoms, nitro group, hydroxy group, amino group, cyano group, which method comprises the steps of
Step (1) treating clarithromycin of Formula II 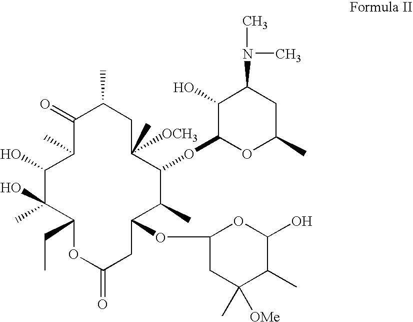
with acid at ambient temperature to give a compound of Formula III 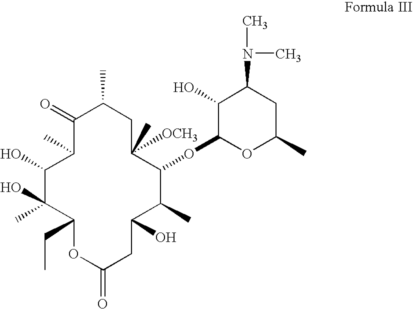
Step (2) reacting the compound of Formula III with a reagent of Formula R′₂O or R′X (wherein R′ is hydroxy protecting group optionally selected from acetyl, benzoyl, butyldiphenylsilyl, methylthiomethyl, methoxy methyl and X is an optional halogen atom) to give a compound of Formula IV 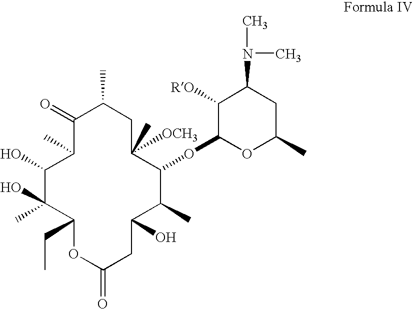
Step (3) reacting the compound of Formula IV with a reagent of Formula R¹COOH, R¹COX, (R¹CO)₂O or R¹COOR⁴ (wherein R¹ is as defined for Formula I in claim 1 and R⁴ is a group selected from pivaloyl group, p-toluenesulfonyl group, isobutoxycarbonyl group, ethoxycarbonyl group or isopropoxycarbonyl group) to give a compound of Formula V 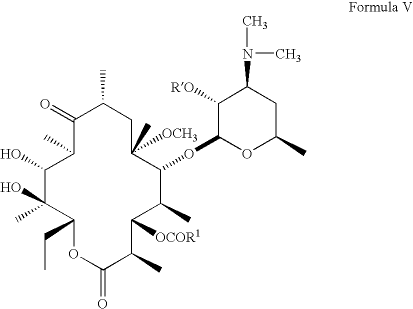
Step (4) desmethylating at 3′-N-dimethyl group of the compound of Formula V with N-iodosuccinamide and acetonitrile or iodine in presence of sodium acetate followed by quench with sodium thiosulphate to obtain the compound of Formula XIII 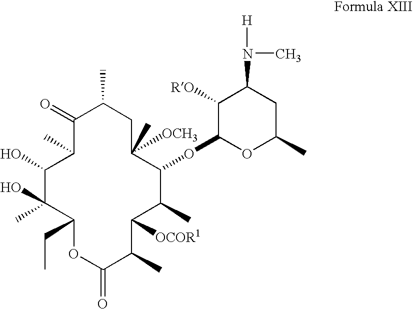
Step (5) treating the compound of Formula XIII with aqueous alcohol to give a compound of Formula VII 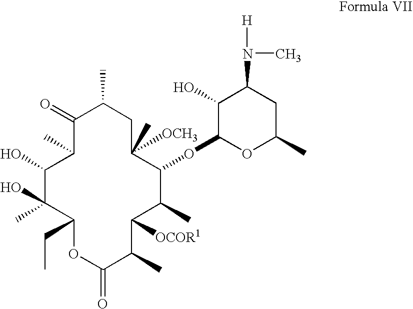
Step (6) reacting the compound of Formula VII with a reagent of Formula R²CHO or R²₂CO (wherein R² is as defined for Formula 1 in claim 1) to give a compound of Formula I 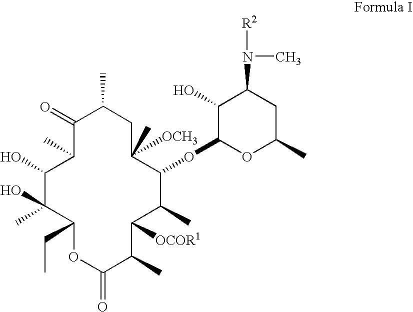
R³═R″═CH₃, R′═H, U═V═OH, and Y=Z=O

29. The process according to claim 28 wherein, the reaction of clarithromycin of Formula II with hydrochloric or dichloroacetic acid to give a compound of Formula III is carried out in presence of aqueous alcohol selected from the group comprising of aqueous methanol, aqueous ethanol, aqueous propanol and aqueous isopropanol.

30. The process according to claim 28 wherein, the reaction of compound of Formula III with a reagent of Formula R′₂O or R′X to give a compound of Formula IV is carried out in presence of an inorganic base selected from the group comprising of sodium hydrogen carbonate, potassium carbonate or an organic base selected from the group comprising of triethylamine, pyridine, tributylamine and 4-N-dimethylaminopyridine.

31. The process according to claim 28 wherein, the reaction of compound of Formula III with a reagent of Formula R′₂O or R′X to give a compound of Formula IV is carried out in presence of an inert solvent selected from the group comprising of dichloromethane, dichloroethane, acetone, ethyl acetate and tetrahydrofuran.

32. The process according to claim 28 wherein, the reaction of compound of Formula IV with a reagent of Formula R¹COOH, R¹COX, (R¹CO)₂O or R¹COOR⁴ to give a compound of Formula V is carried out in presence of an activating agent selected from the group comprising of dichlorohexylcarbodiimide (DCC) and 1-ethyl-3(3-dimethylaminopropyl)carbodiimide hydrochloride (EDCI).

33. The process according to claim 28 wherein, the reaction of compound of Formula IV with a reagent of Formula R¹COOH, R¹COX, (R¹CO)₂O or R¹COOR⁴ to give a compound of Formula V is carried out in presence of an inorganic base selected from the group comprising of sodium hydrogen carbonate, potassium carbonate or organic base selected from the group comprising of triethylamine, pyridine, tributylamine and 4-dimethylaminopyridine.

34. The process according to claim 28 wherein, the reaction of compound of Formula IV with a reagent of Formula R¹COOH, R¹COX, (R¹CO)₂O or R¹COOR⁴ to give a compound of Formula V is carried out in presence of an inert solvent selected from the group comprising of dichloromethane, dichloroethane, acetone, ethyl acetate and tetrahydrofuran.

35. The process according to claim 28 wherein, the reaction of compound of Formula XIII is carried out with aqueous alcohol selected from the group comprising of aqueous methanol, aqueous ethanol, aqueous propanol and aqueous isopropanol to give a compound of Formula VII.

36. The process according to claim 28 wherein, the reaction of the compound of Formula VII with a reagent of Formula R²CHO or R²₂CO to give a compound of Formula I is carried out in presence of a reducing agent selected from the group comprising of sodium borohydride, sodium cyanoborohydride, sodium triacetoborohydride or palladium/carbon catalyst.

37. The process according to claim 28 wherein, the reaction of the compound of Formula VII with a reagent of Formula R²CHO or R²₂CO to give a compound of Formula I is carried out in presence of a protic or non-protic solvent selected from the group comprising of hexane, toluene, methylene chloride, ethylene chloride, chloroform, tetrahydrofuran, N-methyl-pyrrolidinone, diethyl ether, bis-methoxymethyl ether, dimethylformamide, acetonitrile, acetone and ethyl acetate.

38. A process for preparing a compound of Formula I 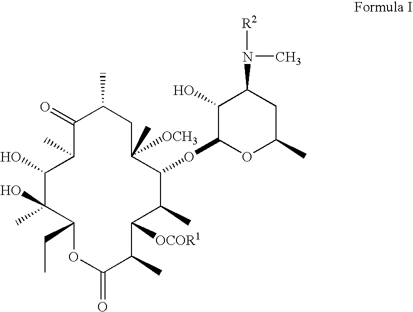
and its pharmaceutically acceptable acid addition salts, pharmaceutically acceptable solvates, enantiomers, diastereomers, polymorphs and metabolites, wherein
R³═R″═CH₃, R′═H, U═V═OH, and Y=Z=O
R¹ represents lower alkyl (C₁-C₅) group, lower alkyl (C₁-C₅) having one or more halogen (F, Cl, Br, I) atoms as substituent (s), lower alkyl (C₁-C₅) amino group, lower alkyl amino (C₁-C₅) carbonyl group, lower alkoxy group (C₁-C₅), five or six membered aryl or heteroaryl ring having 1 to 3 hetero atoms selected from the group consisting of oxygen, nitrogen, sulphur, the aryl or heteroaryl ring may be unsubstituted or substituted by 1 to 3 substituent independently selected from the group consisting of lower alkyl (C₁-C₅) group, lower alkyl (C₁-C₅) group having one or more halogen (F, Cl, Br, I) atoms, lower alkoxy (C₁-C₅) groups, lower alkyl (C₁-C₅) amino group, halogen atoms (F, Cl, Br, I), amino group, nitro group, hydroxy group, cyano group;
R² is selected from C₁-C₆ alkyl group optionally substituted with halogen atoms (F, Cl, Br, I), cycloalkyl (C₃-C₇) group, five to six membered aryl or heteroaryl ring having 1 to 3 hetero atom independently selected from the group consisting of nitrogen, oxygen, sulphur, the aryl or heteroaryl ring may be unsubstituted or substituted by 1 to 3 substituent independently selected from the group consisting of lower alkyl (C₁-C₃), lower alkyl (C₁-C₃) group having one or more halogen (F, Cl, Br, I) atom as substituent (s), lower alkoxy (C₁-C₃) group, lower alkyl (C₁-C₃) amino group, halogen (F, Cl, Br, I) atoms, nitro group, hydroxy group, cyano group, C₁-C₆ alkyl group may also be substituted by a group consisting of NHCOR⁵, NHCOOR⁵, OCOR⁵, COR⁵ [wherein R⁵ represents lower alkyl (C₁-C₅), five to six membered aryl or heteroaryl ring having 1 to 3 hetero atom independently selected from the group consisting of nitrogen, oxygen, sulphur, the aryl or heteroaryl ring may be unsubstituted or substituted by 1 to 3 substituent independently selected from the group consisting of lower alkyl (C₁-C₃), lower alkyl (C₁-C₃) group having one or more halogen (F, Cl, Br, I) atoms as substituent (s), lower alkoxy (C₁-C₃) group, lower alkyl (C₁-C₃) amino group, halogen (F, Cl, Br, I) atoms, nitro group, hydroxy group, cyano group]; C₂-C₆ alkenyl or alkyne group optionally substituted with halogen (F, Cl, Br, I) atoms or a group consisting of NHCOR⁵, NHCOOR⁵, COR⁵, OCOR⁵ (wherein R⁵ is as defined above); cycloalkyl (C₃-C₇) group; five or six membered aryl or heteroaryl ring having 1 to 3 hetero atom independently selected from the group consisting of nitrogen, oxygen, sulphur, the aryl or heteroaryl ring may be unsubstituted or substituted by 1 to 3 substituents independently selected from the group consisting of lower alkyl (C₁-C₃) group, lower alkyl (C₁-C₃) group having one or more halogen (F, Cl, Br, I) atoms as substituent (s), lower alkoxy (C₁-C₃) group, lower alkyl (C₁-C₃) amino, halogen (F, Cl, Br, I) atoms, nitro group, hydroxy group, amino group, cyano group, which method comprises the steps of
Step (1) treating clarithromycin of Formula II 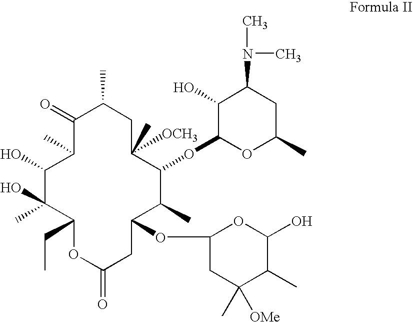
with acid at ambient temperature to give a compound of Formula III 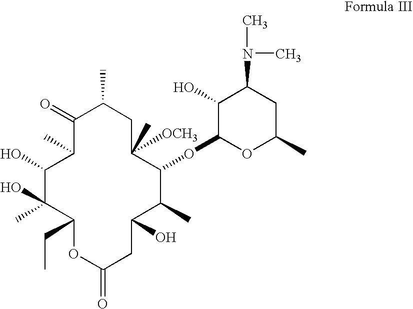
Step (2) reacting the compound of Formula III with a reagent of Formula R′₂O or R′X (wherein R′ is hydroxy protecting group optionally selected from acetyl, benzoyl, butyldiphenylsilyl, methylthiomethyl, methoxy methyl and X is an optional halogen atom) to give a compound of Formula IV 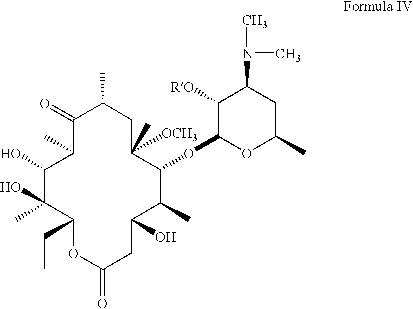
Step (3) desmethylating at 3′-N-dimethyl group of the compound of Formula IV with N-iodosuccinamide and acetonitrile or iodine in presence of sodium acetate followed by quench with sodium thiosulphate to give a compound of Formula XIV 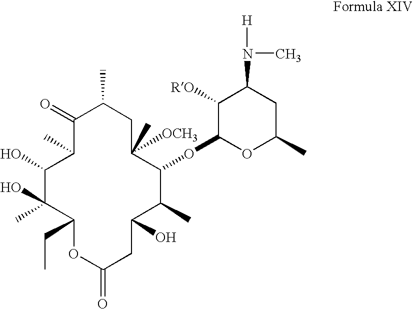
Step (4) reacting the compound of Formula XIV with a reagent of Formula R²CHO or R²₂CO (wherein R² is as defined for Formula 1) to give a compound of Formula XI 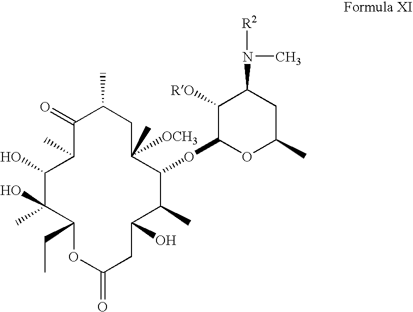
Step (5) reacting the compound of Formula XI with a reagent of Formula R¹COOH, R¹COX, (R¹CO)₂O or R¹COOR⁴ (wherein R¹ is as defined for Formula I and R⁴ is a group selected from pivaloyl group, p-toluenesulfonyl group, isobutoxycarbonyl group, ethoxycarbonyl group or isopropoxycarbonyl group) to give a compound of Formula XII 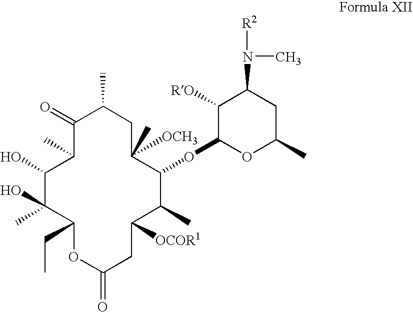
Step (6) treating the compound of Formula XII with aqueous alcohol to give the compound of Formula I 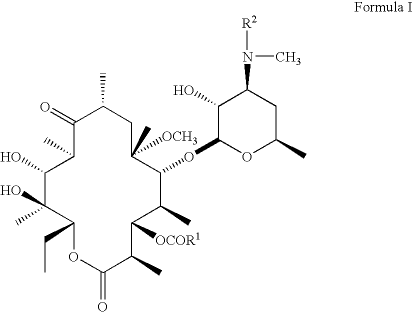
R³═R″═CH₃, R′═H, U═V═OH, and Y=Z=O

39. The process according to claim 38 wherein, the reaction of clarithromycin of Formula II with hydrochloric or dichloroacetic acid to give a compound of Formula III is carried out in presence of aqueous alcohol selected from the group comprising of aqueous methanol, aqueous ethanol, aqueous propanol and aqueous isopropanol.

40. The process according to claim 38 wherein, the reaction of compound of Formula III with a reagent of Formula R′₂O or R′X to give a compound of Formula IV is carried out in presence of an inorganic base selected from the group comprising of sodium hydrogen carbonate, potassium carbonate or an organic base selected from the group comprising of triethylamine, pyridine, tributylamine and 4-N-dimethylaminopyridine.

41. The process according to claim 38 wherein, the reaction of compound of Formula III with a reagent of Formula R′₂O or R′X to give a compound of Formula IV is carried out in presence of an inert solvent selected from the group comprising of dichloromethane, dichloroethane, acetone, ethyl acetate and tetrahydrofuran.

42. The process according to claim 38 wherein, the reaction of the compound of Formula XIV with a reagent of Formula R²CHO or R²₂CO to give a compound of Formula XI is carried out in presence of a reducing agent selected from the group comprising of sodium borohydride, sodium cyanoborohydride, sodium triacetoborohydride or palladium/carbon catalyst.

43. The process according to claim 38 wherein, the reaction of the compound of Formula XIV with a reagent of Formula R²CHO or R²₂CO to give a compound of Formula XI is carried out in presence of a protic or non-protic solvent selected from the group comprising of hexane, toluene, methylene chloride, ethylene chloride, chloroform, tetrahydrofuran, N-methyl-pyrrolidinone, diethyl ether, bis-methoxymethyl ether, dimethylformamide, acetonitrile, acetone and ethyl acetate.

44. The process according to claim 38 wherein, the reaction of compound of Formula XI with a reagent of Formula R¹COOH, R¹COX, (R¹CO)₂O or R¹COOR⁴ to give a compound of Formula XII is carried out in presence of an activating agent selected from the group comprising of dichlorohexylcarbodiimide (DCC) and 1-ethyl-3(3-dimethylaminopropyl)carbodiimide hydrochloride (EDCI).

45. The process according to claim 38 wherein, the reaction of compound of Formula XI with a reagent of Formula R¹COOH, R¹COX, (R¹CO)₂O or R¹COOR⁴ to give a compound of Formula XII is carried out in presence of an inorganic base selected from the group comprising of sodium hydrogen carbonate, potassium carbonate or organic base selected from the group comprising of triethylamine, pyridine, tributylamine and 4-dimethylaminopyridine.

46. The process according to claim 38 wherein, the reaction of compound of Formula XI with a reagent of Formula R¹COOH, R¹COX, (R¹CO)₂O or R¹COOR⁴ to give a compound of Formula XII is carried out in presence of an inert solvent selected from the group comprising of dichloromethane, dichloroethane, acetone, ethyl acetate and tetrahydrofuran.

47. The process according to claim 28 wherein, the reaction of compound of Formula XII is carried out with aqueous alcohol selected from the group comprising of aqueous methanol, aqueous ethanol, aqueous propanol and aqueous isopropanol to give a compound of Formula I.
